(19) United States
(12) Patent Application Publication
(10) Pub. No.: US 2014/0303216 A1
(43) Pub. Date: Oct. 9, 2014

(54) COMPOSITIONS AND METHODS FOR INDUCING DISRUPTION OF BLOOD VASCULATURE AND FOR REDUCING ANGIOGENESIS

(71) Applicant: BOARD OF REGENTS, THE UNIVERSITY OF TEXAS SYSTEM, Austin, TX (US)

(72) Inventors: Edward Marcotte, Austin, TX (US); Hye Ji Cha, Austin, TX (US); John Wallingford, Austin, TX (US)

(21) Appl. No.: 14/355,727

(22) PCT Filed: Nov. 2, 2012
(86) PCT No.: PCT/US2012/063251

§ 371 Date: May 1, 2014

Related U.S. Application Data
(60) Provisional application No. 61/555,212, filed on Nov. 3, 2011.

(51) Int. Cl. A61K 31/427

(52) U.S. Cl. CPC A61K 31/427
USPC 514/365

(57) ABSTRACT

The invention provides methods for reducing angiogenesis and/or inducing vascular disruption in a tissue in a mammalian subject in need thereof, comprising administering to the subject a therapeutic amount of thiabendazole (TBZ) that is effective to reduce angiogenesis and/or induce vascular disruption in the tissue.

This application claims priority to co-pending U.S. provisional Application Ser. No. 61/555,212, filed on Nov. 03, 2011 which is herein incorporated by reference in its entirety for all purposes.

### GOVERNMENT SUPPORT

This invention was made with government support under contract RO1 GM088624-01 awarded by the National Institutes of Health (NIH). The government has certain rights in the invention.

### FIELD OF THE INVENTION

The invention provides methods for inducing disruption of blood vasculature and/or reducing angiogenesis in a tissue in a mammalian subject in need thereof, comprising administering to the subject a therapeutic amount of thiabendazole (TBZ) that is effective to induce disruption of blood vasculature and/or reduce angiogenesis in the tissue.

### BACKGROUND OF THE INVENTION

Angiogenesis, the process of forming new blood vessels, plays an essential role in development, reproduction and tissue repair¹. Because the vascular network supplies oxygen and nutrients to cancer cells as well as to normal cells, angiogenesis also governs the growth of many types of tumors, and is central to malignancy)¹⁻⁴ and to other diseases associated with increased angiogenesis. Angiogenesis is thus considered to be a major therapeutic target for drug development. Some cancers, such as the most common and deadly brain neoplasm, glioblastoma multiformae⁵, are heavily vascularized, but have not responded to current angiogenesis inhibitors^{6,7}.

Because new agents that target the vasculature would increase the arsenal for battling cancers resistant to current therapies¹⁻⁴, there remains a need for novel compositions and methods to their identification.

### SUMMARY OF THE INVENTION

The invention provides a method for inducing disruption of vasculature in a tissue in a mammalian subject in need thereof, comprising administering to said subject a therapeutic amount of thiabendazole (TBZ) that is effective to induce disruption of vasculature in said tissue. In one embodiment, the administration is oral, and said therapeutic amount of TBZ is 200 mg/kg body weight or less. In another embodiment, the therapeutic amount of TBZ is 100 mg/kg body weight or less. In a further embodiment, the administration is oral, and said therapeutic amount of TBZ is from more than 1 mg/kg body weight to 200 mg/kg body weight. In a particular embodiment, the therapeutic amount of TBZ does not substantially increase birth defects in said subject. In yet another embodiment, the therapeutic amount of TBZ does not cause mutations in said subject. In a further embodiment, the therapeutic amount of TBZ does not cause birth defects in the offspring of said subject. In another embodiment, the subject has a disease associated with increased angiogenesis in said tissue compared to normal tissue lacking said disease. In one embodiment, the disease comprises cancer. In a particular embodiment, the cancer comprises cancer cells that do not express a tumor suppressor gene. In one embodiment, the administering reduces one or more symptoms of said disease.

The invention also provides a method for reducing angiogenesis in a tissue in a mammalian subject in need thereof, comprising administering to said subject a therapeutic amount of thiabendazole (TBZ) that is effective to reduce angiogenesis in said tissue. In one embodiment, the administration is oral, and said therapeutic amount of TBZ is 200 mg/kg body weight or less. In another embodiment, the therapeutic amount of TBZ is 100 mg/kg body weight or less. In a further embodiment, the administration is oral, and said therapeutic amount of TBZ is from more than 1 mg/kg body weight to 200 mg/kg body weight. In a particular embodiment, the therapeutic amount of TBZ does not substantially increase birth defects in said subject. In yet another embodiment, the therapeutic amount of TBZ does not cause mutations in said subject. In a further embodiment, the therapeutic amount of TBZ does not cause birth defects in the offspring of said subject. In another embodiment, the subject has a disease associated with increased angiogenesis in said tissue compared to normal tissue lacking said disease. In one embodiment, the disease comprises cancer. In a particular embodiment, the cancer comprises cancer cells that do not express a tumor suppressor gene. In one embodiment, the administering reduces one or more symptoms of said disease.

The invention further provides a method for inducing disruption of vasculature in a tissue in a mammalian subject in need of inducing disruption of vasculature in said tissue, comprising administering to said subject a therapeutic amount of thiabendazole (TBZ) that is effective to induce disruption of vasculature in said tissue. In one embodiment, said administration is oral, and said therapeutic amount of TBZ is 200 mg/kg body weight or less. In a further embodiment, said therapeutic amount of TBZ is 100 mg/kg body weight or less. In yet another embodiment, said administration is oral, and said therapeutic amount of TBZ is from more than 1 mg/kg body weight to 200 mg/kg body weight. In a preferred embodiment, said therapeutic amount of TBZ does not cause mutations in said subject. In another preferred embodiment, said therapeutic amount of TBZ does not cause birth defects in the offspring of said subject. In a particular embodiment, said subject has a disease associated with increased angiogenesis in said tissue compared to normal tissue lacking said disease. In a more preferred embodiment, said disease comprises cancer. In one embodiment, cancer comprises cancer cells that do not express a tumor suppressor gene. In a further embodiment, said administering reduces one or more symptoms of said disease. In yet another embodiment, said therapeutic amount is not fungicidal and is not parasiticidal and is not bactericidal in one or more tissue of said subject.

The invention further provides a method for reducing angiogenesis in a tissue in a mammalian subject in need of reducing angiogenesis in said tissue, comprising administering to said subject a therapeutic amount of thiabendazole (TBZ) that is effective to reduce angiogenesis in said tissue. In one embodiment, said administration is oral, and said therapeutic amount of TBZ is 200 mg/kg body weight or less. In a further embodiment, said therapeutic amount of TBZ is 100 mg/kg body weight or less. In one preferred embodiment, said administration is oral, and said therapeutic amount of TBZ is from more than 1. mg/kg body weight to 200 mg/kg body weight. In one embodiment, said therapeutic amount of TBZ does not cause mutations in said subject. In a particular embodiment, said therapeutic amount of TBZ does not cause birth defects in the offspring of said subject. In a further embodiment, said subject has a disease associated with increased angiogenesis in said tissue compared to normal tissue lacking said disease. In a particular embodiment, said disease comprises cancer. In a further embodiment, said cancer comprises cancer cells that do not express a tumor suppressor gene. In one embodiment, said administering reduces one or more symptoms of said disease. In yet another embodiment, said therapeutic amount is not fungicidal and is not parasiticidal and is not bactericidal in one or more tissue of said subject.

The invention also provides a method for reducing one or more symptoms of cancer in a tissue in a mammalian subject in need thereof, comprising administering to said subject a therapeutic amount of thiabendazole (TBZ) that is effective to reduce one or more symptoms of cancer in said tissue.

### BRIEF DESCRIPTION OF THE DRAWINGS

FIG. 1. Overview of the evolutionary method used to discover a novel vascular disrupting agent. Strict reliance on the evolutionary conservation of the relevant gene module allowed for an experimental design exploiting the unique experimental advantages of each model organism, thus speeding the search for novel angiogenesis inhibitors.

FIG. 2. Identification of candidate angiogenesis inhibitors based upon genetic interactions with a yeast gene module. (A) Summary of the gene module (modified from ref. 8). Tests of genes associated with the yeast phenotype (lovastatin sensitivity) correctly identified novel angiogenesis genes, as in ref. 8 and additionally shown in (B) for the gene rab11b. Morpholino (MO) knockdown of rab11B induces vascular defects in developing Xenopus laevis (frog) embryos, measured by in situ hybridization versus marker gene erg. ISV, intersomitic vein; PCV, posterior cardinal vein; VV, vitellin vein. (C) In an unbiased hierarchical clustering of compounds by their synthetic genetic interaction profiles with yeast genes (analyzing data from ref. 17), the action of TBZ is among those interacting with this gene module and also most similar to lovastatin, the signature compound affiliated with the angiogenesis gene module; hence, TBZ is a likely candidate angiogenesis inhibitor. Here, complete linkage clustering employing uncentered correlation coefficients is shown; additional clustering methods are illustrated in FIG. S2.

FIG. 3. TBZ inhibits angiogenesis in vivo in Xenopus embryos. Formation of Xenopus embryo veins is disrupted, marked by expression of vascular reporter genes (A, B) erg and (C, D) apInr, contrasting treatment with 1% DMSO control only (A, C) with 1% DMSO, 250 μM TBZ, treated at stage 31 and imaged at stages 35-36 (B, D). PCV, posterior cardinal vein; ISV, intersomitic vein; VV, vitellin veins. Similarly, TBZ disrupts vasculature imaged within a living Xenopus embryo and visualized by vascular specific GFP in kdr:GFP frogs from ref. 12, contrasting the vasculature of stage 46 animals treated from stage 41 with the 1% DMSO control (E) or 1% DMSO, 250 μM TBZ (F). Scale bar, 200 μM.

FIG. 4. TBZ significantly disrupts tube formation in cultured human umbilical vein endothelial cells (HUVECs), an in vitro capillary model. Here, we show effects of 1% DMSO-treated control (A) versus 1% DMSO, 100 μM TBZ (B) and 1% DMSO, 250 μM TBZ (C). Scale bar, 100 μm. (D) Tube disruption is dose-dependent and comparable to that from silencing known pro-angiogenic gene HOXA9.

FIG. 5. TBZ disrupts newly established vasculature, as visualized in vivo using time-lapse fluorescence microscopy within kdr:GFP frogs. Retraction and rounding of vascular endothelial cells (arrowheads) is apparent in TBZ-treated embryos (A, time lapse of frogs treated as in FIG. 3) as compared with continued vascular growth in control animals (FIG. S7). Scale bar, 80 μm. (B) A series with intermediate time points is shown for a sub-region of that shown in (A). (B′) Schematics of the images in (B) indicate positions of specific cells.

FIG. 6. After disassembly of the vasculature by TBZ, washout of the drug leads to partial recovery of the vascular network. Two time series are shown in (A) and (B), imaged as in FIG. 5. Following washout, cells dissociated by TBZ treatment recommence cell elongation and connection. Individual cells are indicated by arrows/asterisks. Scale bar in (A), 50 μm; in (B), 40 μm.

FIG. 7. TBZ impedes migration of HUVECs in a wound scratch assay, but treatment with the Rho Kinase inhibitor Y27632 reverses TBZ's effects. (A) The effects of 1% DMSO-treated control versus 1% DMSO, 250 μM TBZ, and 1% DMSO, 250 μM TBZ, 10 μM Y27632. Scale bar, 200 μm. (B) quantifies the dose-dependent suppression of TBZ inhibition by Y27632. Error bars represent the mean±1 s.d. across 3 wells (1 of 3 trials). TBZ results in disorganization of actin stress fibers, as shown in (C) for 1% DMSO-treated control versus 1% DMSO, 250 μM TBZ-treated cells. Scale bar, 20 μm.

FIG. 8. TBZ slows the growth of human HT1080 fibrosarcoma xenograft tumors in athymic Cre nu/nu mice. Tumors are significantly reduced in size in TBZ-treated animals (A), shown in (B) biopsied from mice after 27 d of 50 mg/kg (corresponding to 250 μM) TBZ treatment, and quantified in (C) and (D) (1 of 2 trials).

FIG. 9. Blood vessel density is significantly reduced within TBZ-treated tumors. (A) and (B) show tumor vasculature visualized by immunohistochemistry of microdissected tumor sections using an anti-CD31 (PECAM-1) antibody staining for vasculature in the region of highest vessel density (“hot spots”; scale bar, 100 μm), and the total area of PECAM-1 staining above a fluorescence intensity threshold (arbitrary units) is quantified in (C).

FIG. 10. Unilateral morpholino (MO) knockdown of rab11b induces vascular defects in developing Xenopus laevis (frog) embryos, showing the control versus knockdown sides of the same animal and measured by in situ hybridization versus marker gene apinr. ISV, intersomitic vein; PCV, posterior cardinal vein.

FIG. 11. In yeast chemical genetic interaction datasets ref. 17, TBZ treatment consistently clustered with lovastatin treatment across different choices of similarity measures and clustering algorithms. Three cases out of 19 trials are illustrated here, organized as in FIG. 2C.

FIG. 12. TBZ inhibits ectopic angiogenesis (note the doubled PCV in the left panel) stimulated by Affy-gel blue beads (75-150 μm diameter, indicated by white arrowheads) pre-soaked with 0.7 mg/ml vascular endothelial growth factor (VEGF) and microsurgically implanted into developing Xenopus embryos, assaying for the vasculature by ISH versus erg or apinr (showing data for erg). 6 of 8 control animals developed ectopic PCV or VV, as opposed to 0 of 9 TBZ-dosed animals (p value=0.0023). TBZ-treated embryos show notably disorganized (cellularized) vasculatures.

FIG. 13. Somitic muscle, defined with the 12/101 antibody, on 1% DMSO, 250 μM, TBZ-treated Xenopus embryo, is normal compared to 1% DMSO control. Both were treated at stage 31 and imaged at stages 35-36.

FIG. 14. Tests of commercially available TBZ variants indicate that in vivo angiogenesis inhibition activity in Xenopus varies strongly across benzimidazoles and suggests necessary chemical moieties (for example, suggesting that the thiazole group, or at least its nitrogen, is important to activity).

FIG. 15. TBZ treatment shortly after the posterior cardinal vein (PCV) is established (stage 36) causes Xenopus vascular structures to re-cellularize in vivo, shown by in situ hybridization versus apinr at stage 39.

FIG. 16. Blood vessel development visualized in vivo using time-lapse fluorescence microscopy of the vasculature developing within a living Xenopus embryo. Arteries and veins are visualized as in FIGS. 3 and 5 by vascular-specific kdr:GFP frogs from ref. 12, showing the vasculature of stage 46 animals treated from stage 41 with the 1% DMSO control. Scale bar, 80 μm.

FIG. 17. Retraction and rounding of vascular endothelial cells (blue arrowheads) is apparent at higher magnification in TBZ-treated embryos (as in FIGS. 3 and 5). Scale bar, 30 μm.

FIG. 18. TBZ treatment (A) increases apoptosis and (B) decreases proliferation of HUVEC cells cultured on 0.1% gelatin, but only by approximately 2-fold. Both changes are significant under a t test (p=0.015, p=0.01, respectively). Error bars represent mean±1 s.d. across 14-16 fields of view of 200× magnification confocal microscopy cell images across 2-3 independent experiments. Scale bar, 100 μm.

FIGS. 19.(A, B) GGPP does not reverse the impeded migration of HUVECs in a wound-scratch assay. (A) shows effects of 1% DMSO-treated control versus 1% DMSO, 250 μM TBZ, and 1% DMSO, 250 μM TBZ, 25 μM GGPP. (B) shows quantification as a function of varying GGPP concentrations. Error bars represent mean±1 s.d. across 3 wells (1 of 2 trials). Scale bar, 200 μm.

FIG. 20. lmmunohistochemical analysis of β-tubulin does not show a definite distinction between 1% DMSO-treated control and 1% DMSO, 250 μM TBZ-treated HUVECs (A), but tubulins in HUVECs identified by a quantitative mass-spectroscopy analysis were significantly reduced with TBZ treatment (B). Scale bar in (A), 20 μm.

FIG. 21. TBZ treatment does not significantly affect the proliferation of HT1080 cells. Error bars represent mean±1 s.d. across 14-15 fields of view of 200× magnification confocal microscopy cell images across 2 independent experiments. Scale bar, 100 μm.

FIG. 22. TBZ does not significantly alter intracellular or secreted VEGF levels in HT1080 cells. VEGF levels in HT1080 cells and conditioned medium were measured in 1% DMSO-treated control and 1% DMSO, 250 μM TBZ-treated HT1080 cells, assaying cellular VEGF by Western blotting (A) and secreted VEGF by ELISA (B). Glyceraldehyde-3-phosphate dehydrogenase (GAPDH) level was measured for a Western blotting control.

### DEFINITIONS

To facilitate understanding of the invention, a number of terms are defined below.

“Angiogenesis” means the formation of new blood vessels.

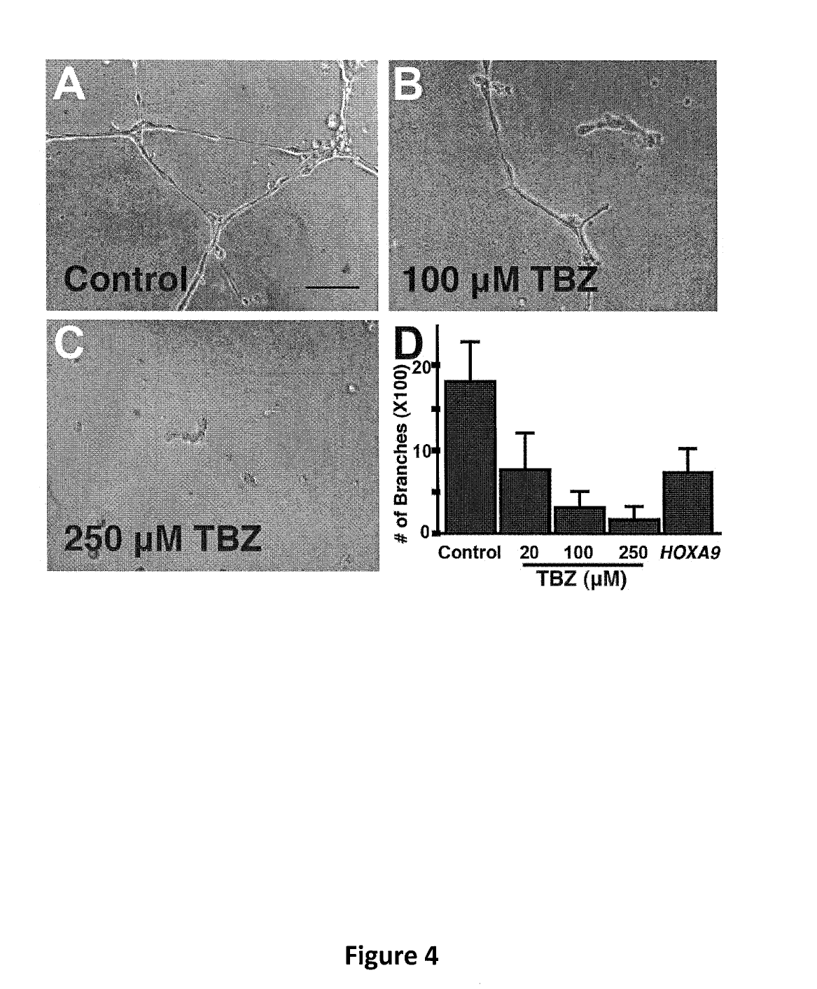

“Disruption of vasculature” refers to disintegration (i.e., reduced number) of pre-existing blood vessels (exemplified in FIGS. 4A; 17), and /or disassembly of vascular endothelial cell-cell adhesion (exemplified in Example 9).

“Blood vessel” includes artery, vein, and capillary.

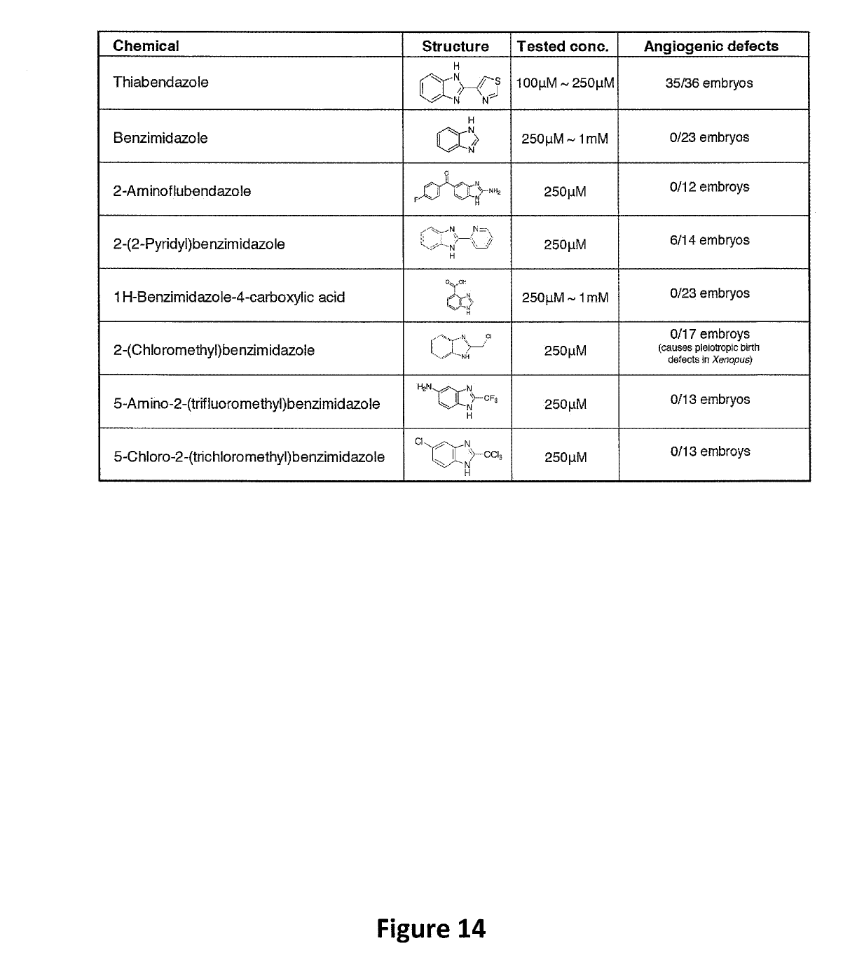

“Thiabendazole” and “TBZ” interchangeably refer to 4-(1H-1,3-benzodiazol-2-yl)-1,3-thiazole shown in FIG. 14. TBZ was initially marketed by Merck as MINTEZOL™, and is now off-patent and produced as a generic drug under the trade names APL-LUSTER™, MERTECT™, MYCOZOL™, TECTO™, TRESADERM™, and ARBOTECT™. TBZ is a fungicide and parasiticide, and has been used by humans since its FDA approval in 1967.

“Benzimidazole” refers to a large chemical family used to treat nematode and trematode infections. Benzimidazoles are exemplified by mebendazole, flubendazole, fenbendazole, oxfendazole, oxibendazole, albendazole, albendazole sulfoxide, thiabendazole, thiophanate, febantel, netobimin, and triclabendazole.

“Therapeutic amount,” “pharmaceutically effective amount,” and “therapeutically effective amount,” are used interchangeably herein to refer to an amount that is sufficient to achieve a desired result, whether quantitative and/or qualitative. In a preferred embodiment, the therapeutic amount induces disruption of blood vasculature and/or reduces angiogenesis in a tissue and/or reduces one or more disease symptoms in angiogenic tissue.

“Parasiticide” and grammatical equivalents, when in reference to the amount of a compound (such as TBZ), refer to an amount that reduces in the level of infection of a tissue by a parasite, such as nematode and/or trematode, as exemplified by Ascaris lumbricoides (“common roundworm”), Strongyloides stercoralis (threadworm), Necator americanus, Ancylostoma duodenale (hookworm), Trichuris trichiura (whipworm), Ancylostoma braziliense (dog and cat hookworm), Toxocara canis, Toxocara cati (ascarids), and Enterobius vermicularis (pinworm).

“Fungicidal” and grammatical equivalents, when in reference to the amount of a compound (such as TBZ), refer to an amount that reduces in the level of infection of a tissue by a fungus, such as mold, blight, rot blight, and stain.

“Bactericidal” and grammatical equivalents, when in reference to the amount of a compound (such as TBZ), refer to an amount that reduces in the level of infection of a tissue by a bacterium.

The term “apoptosis” means non-necrotic cell death that takes place in metazoan animal cells following activation of an intrinsic cell suicide program. Apoptosis is a normal process in the development and homeostasis of metazoan animals. Apoptosis involves characteristic morphological and biochemical changes, including cell shrinkage, zeiosis, or blebbing, of the plasma membrane, and nuclear collapse and fragmentation of the nuclear chromatin, at intranucleosomal sites. During apoptosis, cells undergo various changes that result in the eventual lysis of the cell into apoptotic bodies which are then typically phagocytosed by other cells. One of skill in the art appreciates that reducing the level of apoptosis results in increased cell survival, without necessarily (although it may include) increasing cell proliferation.

“Vascular disrupting agent” and “VDA” are interchangeably used herein to refer to compounds that break down existing vascular structures, thereby disrupting blood flow, particularly within solid tumors (Heath, V. L. & Bicknell, R. Anticancer strategies involving the vasculature. Nat Rev Clin Oncol 6, 395-404 (2009); Tozer, G. M., Kanthou, C. & Baguley, B. C. Disrupting tumour blood vessels. Nat Rev Cancer 5, 423-35 (2005); and Hinnen, P. & Eskens, F. A. Vascular disrupting agents in clinical development. Br J Cancer 96, 1159-65 (2007)). Several VDAs are in phase II and III trials (Heath et al. (2009)).

“Angiogenesis inhibitors” target newly forming blood vessels, but “vascular disrupting agents” and “VDAs” target already established blood vessels.

“Cancer” refers to a plurality of cancer cells that are undergoing early, intermediate and/or advanced stages of multi-step neoplastic progression as previously described (Pitot et al., Fundamentals of Oncology, 15-28 (1978)). Cancer may or may not be metastatic, such as ovarian cancer, breast cancer, lung cancer, prostate cancer, cervical cancer, pancreatic cancer, colon cancer, stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia.

“Tumor suppressor gene” refers to a gene whose expression (into RNA and/or a polypeptide) in a cell reduces the risk of progression of the cell into a cancer cell. Tumor suppressor genes are exemplified by p53, p16, p21, Rb, p15, BRCA1, BRCA2, zac1, p73, ATM, HIC-1, DPC-4, FHIT, NF2, APC, DCC, PTEN, ING1, NOEY1, NOEY2, PML, OVCA1, MADR2, WT1, 53BP2, IRF-1, MDA-7, C-CAM, CD95, ST5, ST7, ST14, and YPEL3.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence, antibody, etc.), cell, and/or phenomenon (e.g., angiogenesis, disruption of blood vasculature, birth defect, mutation, apoptosis, disease symptom, cell-to-cell adhesion, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first sample (or the first subject) is exemplified by, but not limited to, a sample (or subject) that has been manipulated using the invention's compositions and/or methods. In a further embodiment, the second sample (or the second subject) is exemplified by, but not limited to, a sample (or subject) that has not been manipulated using the invention's compositions and/or methods. In an alternative embodiment, the second sample (or the second subject) is exemplified by, but not limited to, a sample (or subject) that has been manipulated, using the invention's compositions and/or methods, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the invention's compositions and/or methods is sought to be determined on one sample (or subject). In another embodiment, the first and second samples (or subjects) may be different, such as when comparing the effect of the invention's compositions and/or methods on one sample (subject), for example a patient participating in a clinical trial and another individual in a hospital. In some embodiments, reduced mutations and/or birth defects can be shown in a clinical trial. Where it is shown there, the reduction in any subject subsequently is presumed (without the need for a comparison later).

The terms “increase,” “elevate,” “raise,” “induce,” and grammatical equivalents (including “higher,” “greater,” etc.) when in reference to the level of any molecule (e.g., amino acid sequence, and nucleic acid sequence, antibody, etc.), cell, and/or phenomenon (e.g., angiogenesis, disruption of vasculature, birth defect, mutation, apoptosis, disease symptom, cell-to-cell adhesion, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first sample (or the first subject) is exemplified by, but not limited to, a sample (or subject) that has been manipulated using the invention's compositions and/or methods. In a further embodiment, the second sample (or the second subject) is exemplified by, but not limited to, a sample (or subject) that has not been manipulated using the invention's compositions and/or methods. In an alternative embodiment, the second sample (or the second subject) is exemplified by, but not limited to, a sample (or subject) that has been manipulated, using the invention's compositions and/or methods, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the invention's compositions and/or methods is sought to be determined on one sample (or subject). In another embodiment, the first and second samples (or subjects) may be different, such as when comparing the effect of the invention's compositions and/or methods on one sample (subject), for example a patient participating in a clinical trial and another individual in a hospital. In some embodiments, increased mutations and/or birth defects can be shown in a clinical trial. Where it is shown there, the reduction in any subject subsequently is presumed (without the need for a comparison later).

The term “not substantially reduced” when in reference to the level of any molecule (e.g., amino acid sequence, and nucleic acid sequence, antibody, etc.), cell, and/or phenomenon (e.g., angiogenesis, disruption of vasculature, birth defect, mutation, apoptosis, disease symptom, cell-to-cell adhesion, etc.) in a first sample (or in a first subject) relative to a second sample (or relative to a second subject), means that the quantity of molecule, cell and/or phenomenon in the first sample (or in the first subject) is from 91% to 100% of the quantity in the second sample (or in the second subject).

The terms “alter” and “modify” when in reference to the level of any molecule (e.g., amino acid sequence, and nucleic acid sequence, antibody, etc.), cell, and/or phenomenon (e.g., angiogenesis, disruption of vasculature, birth defect, mutation, apoptosis, disease symptom, cell-to-cell adhesion, etc.) refer to an increase and/or decrease in the level of the molecule, cell, and/or phenomenon.

“Substantially the same” when in reference to the level of any molecule (e.g., amino acid sequence, and nucleic acid sequence, antibody, etc.), cell and/or phenomenon molecule (e.g., amino acid sequence, and nucleic acid sequence, antibody, etc.), cell, and/or phenomenon (e.g., angiogenesis, disruption of vasculature, birth defect, mutation, apoptosis, disease symptom, cell-to-cell adhesion, etc.) in a first sample (or in a first subject) relative to a second sample (or relative to a second subject), mean that the quantity of molecule, cell and/or phenomenon in the first sample (or in the first subject) is not different from the quantity in the second sample (or in the second subject) using any art-accepted statistical method of analysis. In one embodiment, the quantity of molecule, cell and/or phenomenon in the first sample (or in the first subject) is from 90% to 100% (e.g., 90%, 91%, 92%, 93%, 94%, 95%, 96%, 97%, 98%, 99%, and 100%) of the quantity in the second sample (or in the second subject).

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### DESCRIPTION OF THE INVENTION

For further clarity, the invention is further described under (A) Methods for Inducing Disruption of Blood Vasculature and/or for Reducing Angiogenesis, and (B) Disease Associated With Increased Angiogenesis.

A. Methods for Inducing Disruption of Blood Vasculature and/or for Reducing Angiogenesis

In one embodiment, the invention provides a method for inducing disruption of blood vasculature and/or for reducing angiogenesis in a tissue in a mammalian subject in need thereof, comprising administering to the subject a therapeutic amount of thiabendazole (TBZ) that is effective to induce disruption of blood vasculature and/or reduce angiogenesis in the tissue.

A systematic analysis of conserved genetic modules has recently shown that genes in yeast that maintain cell walls have been repurposed in vertebrates to regulate vein and artery growth. We reasoned that by analyzing such repurposed modules, we might identify small molecules targeting the yeast pathway that also act as angiogenesis inhibitors suitable for chemotherapy. This insight led to the finding that thiabendazole, an orally-available antifungal drug in clinical use for 40 years, also potently inhibits angiogenesis in animal models and in human cells. Moreover, in vivo time-lapse imaging revealed that thiabendazole reversibly disassembles newly-established blood vessels, marking it as vascular disrupting agent (VDA) and thus as a potential complementary therapeutic for use in combination with current anti-angiogenic therapies. Importantly, we also show that thiabendazole slows tumor growth and decreases vascular density in preclinical fibrosarcoma xenografts. Thus, an exploration of the evolutionary repurposing of gene networks has led directly to the identification of an important new therapeutic application for an inexpensive drug that is already approved for clinical use in humans.

In sum, an analysis of evolutionary repurposing of a genetic module shared from yeast to humans has led directly to the discovery that an orally available drug, thiabendazole, already FDA approved for clinical use in humans, also acts as an angiogenesis inhibitor and vascular disrupting agent. Its more than 40 years of historical safety data, low cost, and generic availability make TBZ a compelling candidate for translation into the clinic as a chemotherapeutic agent, and it is currently the only VDA approved for human use (albeit for a different purpose). This research emphasizes the advantages of an evolutionary approach to drug discovery, in which the natural experimental advantages of different organisms are exploited to accelerate our understanding of a conserved genetic module. Importantly, this approach proved effective even though the associated organismal phenotypes were entirely unrelated. These results suggest that a fundamental understanding of systems biology will prove to be directly relevant to drug discovery, complementing traditional screening approaches to pharmacophore discovery and accelerating both basic and clinical biomedical research.

The art discloses benzimidazole compounds as potential therapeutics (U.S. Pat. No. 7,423,015, U.S. Pat. No. 6,645,950, U.S. Pat. No. 7,064,215, and USPTO Patent Application 2009/0005416) such as for reducing angiogenesis (Horia et al. (2002) Cancer Letters 183: 53-60, Mukhopadhyay et al. (2002) Clinical Cancer Research 8: 2963-2969, Kumar et al. (2001) CANCER RESEARCH 61: 2232-2238). However, the art does not disclose the instant invention.

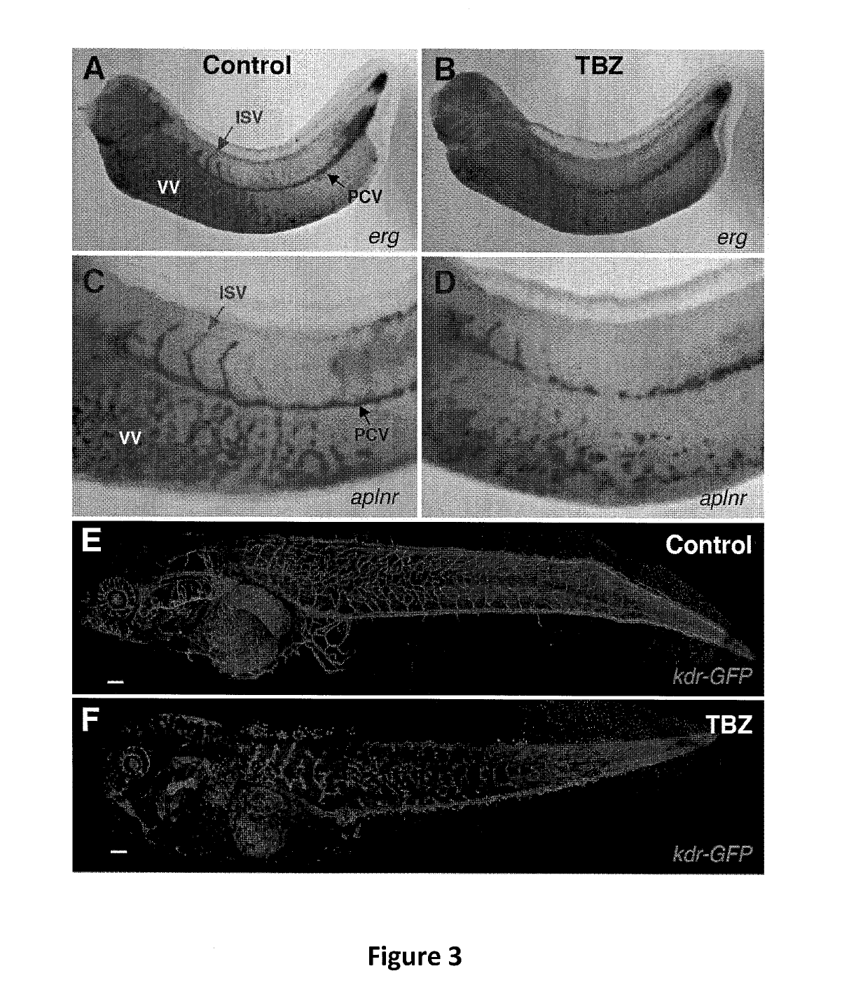

For example, data herein demonstrate that TBZ severely impaired angiogenesis in vitro in human umbilical vein endothelia cells (HUVECs), and in vivo in developing Xenopus embryos (Example 4; FIGS. 3, 4, 5), as well as in human tumor xenografts in mouse (Example 10; FIG. 4D-F).

Mammalian subjects to which the invention's compositions and methods may be applied include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla. The invention's compositions and methods are also useful for a subject “in need of reducing one or more symptoms of” a disease includes a subject that exhibits and/or is at risk of exhibiting one or more symptoms of the disease. For Example, subjects may be at risk based on family history, genetic factors, environmental factors, etc. This term includes animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable). The invention's compositions and methods are also useful for a subject “at risk” for disease refers to a subject that is predisposed to contracting and/or expressing one or more symptoms of the disease. This predisposition may be genetic (e.g., a particular genetic tendency to expressing one or more symptoms of the disease, such as heritable disorders, etc.), or due to other factors (e.g., environmental conditions, exposures to detrimental compounds, including carcinogens, present in the environment, etc.). The term subject “at risk” includes subjects “suffering from disease,” i.e., a subject that is experiencing one or more symptoms of the disease. It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

The invention's compositions may be administered to a mammalian subject. The term “administering” to a subject means providing a molecule to a subject. This may be done using methods known in the art (e.g., Erickson et al., U.S. Pat. No. 6,632,979; Furuta et al., U.S. Pat. No. 6,905,839; Jackobsen et al., U.S. Pat. No. 6,238,878; Simon et al., U.S. Pat. No. 5,851,789), such as in parenteral, oral, intraperitoneal, intranasal, topical and sublingual forms. Parenteral routes of administration include, for example, subcutaneous, intravenous, intramuscular, intrastemal injection, and infusion routes. In one preferred embodiment, administration is oral.

While not intending to limit the maximum therapeutic amount of the invention's TBZ compositions that are useful in the invention's methods, in one embodiment, administration is oral, and the therapeutic amount of TBZ is 1,000 μM or less (i.e., is 200 mg/kg body weight or less. Data herein demonstrate angiogenesis inhibition in both human cells in vitro and in frogs in vivo at a concentration of from 100 to 250 μM (Example 4 & Example 5, FIG. 14). This dose corresponds to from 20 to 50 mg/kg body weight (Example 4, FIGS. 3, 4).

Surprisingly, the dosages of TBZ that are useful in the inventions' methods for reducing angiogenesis were not anti-angiogenic when using other benzimidazoles, such as Benzimidazole, 2-Aminoflubendazole, 1H-Benzimidazole-4-carboxylic acid, 2-(Chloromethyl)benzimidazole, 5-Amino-2-(trifluoromethyl)benzimidazole, and 5-Chloro-2-(trichloromethyl)benzimidazole (FIG. 14). Indeed, benzimidazole was inactive in reducing angiogenesis at doses up to 1 mM and administration of other benzimidazoles caused diverse developmental defects (such as pleiotropic birth defects in Xenopus caused by 250 μM of 2-(Chloromethyl)benzimidazole; FIG. 14) in the absence of angiogenesis inhibition (FIG. 14).

Also, without limiting the maximum therapeutic amount of the invention's TBZ compositions that are useful in the invention's methods, in one embodiment, administration is oral, and the therapeutic amount of TBZ is 500 μM or less (i.e., is 100 mg/kg body weight or less).

While not intending to limit the minimum therapeutic amount of the invention's TBZ compositions that are useful in the invention's methods, in one embodiment, administration is oral, and the therapeutic amount of TBZ is more than 5 (i.e., more than 1 mg/kg body weight). Thus, in one embodiment, a preferred range of therapeutic amount of orally administered TBZ compositions is from more than 5 μM to 1,000 μM. (i.e., from more than 1 mg/kg body weight to 200 mg/kg body weight).

In particular embodiments, the invention's TBZ dosage that is effective in inducing disruption of blood vasculature and/or reducing angiogenesis is lower than the oral LD₅₀ of the TBZ formulation MINTEZOL™ of 1.3 to 3.6 g/kg body weight, 3.1 g/kg body weight, and 3.8 g/kg body weight in the mouse, rat, and rabbit, respectively and that the human approved recommended maximum daily dose is 3 g/kg body weight. As such, one advantage of the invention's TBZ dosages that are effective in inducing disruption of blood vasculature and/or inhibiting angiogenesis is that they have excellent safety data in humans and model animals.

The invention's use of TBZ to induce disruption of blood vasculature and/or reduce angiogenesis has the additional advantage that TBZ has already been approved by the U.S. Food and Drug Administration (FDA) for systemic oral use in humans (as an anti-fungal and anti-helmintic treatment). TBZ was initially marketed by Merck as MINTEZOL™, and is now off-patent and produced as a generic drug under the trade names APL-LUSTER™, MERTECT™, MYCOZOL™, TECTO™, TRESADERM™, and ARBOTECT™. TBZ has been used by humans since its FDA approval in 1967, so its safety has been well-established. In animals, TBZ has no carcinogenic effects in either short-term or long-term studies at doses up to 15 times the usual human dose^{18,19}. Moreover, TBZ does not appear to affect fertility in mice or rats, and it is not a mutagen in standard in vitro microbial mutagen tests, micronucleus tests or host mediated assays in vivo^{18,19}.

In a particular embodiment, the therapeutic amount of TBZ does not increase birth defects in the subject. Data herein show that, surprisingly, TBZ was safer than other benzamides, since benzamides other that TBZ resulted in pleiotropic birth defects at dosages similar to those at which TBZ induced disruption of blood vasculature and/or reduced angiogenesis. For example, data herein demonstrate pleiotropic birth defects in Xenopus caused by 250 μM of 2-(Chloromethyl)benzimidazole (FIG. 14).

In further embodiments, the therapeutic amount of TBZ does not alter endothelial cell apoptosis in the tissue. Surprisingly, while not intending to limit the invention to any particular mechanism, data herein demonstrate that TBZ's anti-angiogenic activity is the result of its reduction in cell-to-cell adhesion, rather than increasing endothelial cell apoptosis (Example 9).

Thus, data herein, (Example 9, FIG. 14) distinguishes the mechanism of action of TBZ from other vascular disrupting agents (VDAs) such as ASA404, which act by inducing endothelial cell apoptosis²³, but which failed to show efficacy in a recent Phase III clinical trial for treatment of lung cancer²⁵.

B. Disease Associated With Increased Angiogenesis

In a preferred embodiment, the invention's compositions and methods are administered to a mammalian subject that has a disease associated with increased angiogenesis in a tissue compared to normal tissue lacking the disease. In a particularly preferred embodiment, administering the invention's compositions reduces one or more symptoms of the disease.

While not intending to limit the type of disease associated with increased angiogenesis, in one embodiment, the disease is selected from the group of cancer, diabetic retinopathy, macular degeneration, psoriasis, hemangioma, gingivitis, rheumatoid arthritis, osteoarthritis, and inflammatory bowel disease.

In a preferred embodiment, the disease comprises cancer. In a more preferred embodiment, the cancer comprises cancer cells that do not express a tumor suppressor gene, such as, without limitation, multiple myeloma, lung cancer, breast cancer, colorectal cancer, and kidney cancer.

### Experimental

The following examples serve to illustrate certain preferred embodiments and aspects of the present invention and are not to be construed as limiting the scope thereof.

### EXAMPLE 1

### Materials And Methods

The following is a brief description of the exemplary materials and methods used in the subsequent Examples.

### Clustering Analysis

Compound genetic interaction profiles were downloaded (Hillenmeyer et al., Science 18 April 2008 Vol. 320 no. 5874 pp. 362-365). We employed the P-values reported for fitness defects in the yeast homozygous deletion collection for all analyses¹⁷. Candidate angiogenesis inhibitors were prioritized that consistently clustered with lovastatin across different choices of similarity measures and hierarchical clustering algorithms, specifically, centered and uncentered correlation, Spearman rank correlation, absolute correlation (centered and uncentered), Euclidean distance, City-block distance, employing centroid linkage, complete linkage, single linkage, or average linkage. Clustering results were visualized with Cluster 3.0 and Java TreeView.

### Xenopus Embryo Manipulations

Female adult Xenopus were ovulated by injections of human chorionic gonadotropin, and eggs were fertilized in vitro and dejellied in 3% cysteine (pH 7.9) and subsequently reared in ⅓×Marc's modified Ringer's (MMR) solution. For microinjections, embryos were placed in a solution of 2% Ficoll in ⅓×MMR solution, injected using forceps and an Oxford universal manipulator, reared in 2% Ficoll in ⅓×MMR to stage 9, then washed and reared in ⅓×MMR solution alone. For bilateral rab11b knock-down experiments, the posterior cardinal vein and intersomitic veins were targeted by injecting Morpholino antisense oligonucleotides (MOs) into the two ventral cells equatorially at the four-cell stage. For unilateral knockdown, only one ventral cell was injected. MOs were injected at 40 ng per blastomere. For the experiments to see the drug effects, embryos were placed in a solution of each chemical dissolved in 1% DMSO diluted in ⅓×MMR during indicated stages. For bead micro-surgery implantation, Affi-Gel Blue Gel beads (Bio-Rad) were soaked with 0.7 mg/ml recombinant mouse VEGF 164 aa (R&D systems) or BSA as a control. Whole-mount in situ hybridization for erg and apinr was performed as described²⁹.

Morpholino Oligonucleotides and cDNA Clones

Erg and apinr cDNAs were obtained from Open BioSystems (erg: IMAGE:5512670, apinr: IMAGE:8321886). Translation blocking antisense morpholinos for rab11b were designed based on the sequences from the National Center for Biotechnology Information database (Accession number: BC082421.1). MOs were obtained from Gene Tools with the following sequence: 5′-CGTATTCGTCATCTCTGGCTCCCAT-3′.

### Cell Culture

Human umbilical vein endothelial cells (HUVECs) were purchased from Clonetics, and were used between passages 4 and 9. HUVECs were cultured on 0.1% gelatin-coated (Sigma) plates in endothelial growth medium-2 (EGM-2; Clonetics) in tissue culture flasks at 37° C. in a humidified atmosphere of 5% CO₂.

### In Vitro Angiogenesis Assays

HUVECs (10⁴ cells) were seeded in a 96-well plate coated with 50 μl of ECMatrix (Chemicon) or Matrigel (BD Bioscience) according to the manufacturer's instructions. Cells were incubated for 16 h on EGM-2 containing thiabendazole, dissolved in 1% DMSO. Negative control cells were treated with 1% DMSO in the same manner. As a positive control, siRNA versus the human HoxA9 sequence³⁰ was transfected into HUVECs using Lipofectamine RNAiMAX (Invitrogen) according to the manufacturer's instructions. Tube formation was observed using an inverted microscope (Nikon, eclipse TS100), and branch points were measured using ImageJ software (http://rsb.info.nih.gov/ij).

### Cell Migration Assays

HUVECs (1.2×10⁵ cells) were seeded into 24-well plate for 24 h, and the monolayers were wounded identically. Then, cells were washed with PBS and treated with EBM-2 containing 1% DMSO or 250 μM TBZ dissolved in 1% DMSO with combination of Y27632 or GGPP (Sigma). In the case of Y27632 treatment, cells were preincubated for 2 h before wounding. Cells were photographed at time zero and after 15 h, and the ratios of cell free area [(0 h-15 h)/0 h] were calculated.

### Xenograft Model

Specific pathogen-free athymic Cre Nu/nu mice were purchased from Charles River Laboratories. The HT-1080 human fibrosarcoma cell line was obtained from the American Type Culture Collection (ATCC). HT-1080 cells were cultured in DMEM (Gibco) containing 10% fetal bovine serum (FBS, Gibco) in tissue culture flasks at 37° C. in a humidified atmosphere of 5% CO₂. In order to generate a mouse xenograft model, a suspension of the HT-1080 cells (3×10⁶ in 50p1 PBS) mixed with an equal volume of Matrigel (BD Bioscience) was subcutaneously implanted into the flank region of 7-8 week old female mice. Upon establishment of tumors (approx. 40 mm³), mice were given intraperitoneal injections of 1 mg thiabendazole (Sigma-Aldrich), suspended in 20 μl 100% DMSO. As a control, an equal volume of DMSO was injected in the same manner. Tumor growth was monitored by measuring the length and width of each tumor using digital calipers, and the tumor volume in mm³ calculated by the formula: Volume=(width)²×length/2. Upon a tumor reaching the maximum size permitted by the Institutional Animal Care and Use Committee (1.5 cm in diameter), the mouse was sacrificed, and the tumor excised.

### Immunohistochemistry

Each tumor was fixed with 4% paraformaldehyde in PBS, and cryostat sections were processed. After blocking with 5% goat serum in PBST (0.3% Triton X-100 in PBS) for 1 h at room temperature, sectioned tissues were incubated with anti-mouse CD31 antibody, hamster clone 2H8, 1:100 (Millipore). After several PBST washes, samples were incubated for 2 h at room temperature with FITC-conjugated anti-hamster IgG antibody, 1:1000 (Jackson ImmunoResearch). In order to determine the effect of thiabendazole on proliferation and apoptosis, 2×10⁵ HUVECs were cultured in 6-well plates and treated with thiabendazole dissolved in 1% DMSO. Control cells received 1% DMSO. After 24 h, cells were fixed using 4% paraformaldehyde in PBS. Cell membranes were permeabilized with 0.2% Triton X-100 in PBS, and nonspecific immunobinding sites were blocked with 5% goat serum for 1 h at room temperature. Cells were incubated with primary antibodies to Caspase-3, rabbit polyclonal (Abcam) or Phospho-histone H3 (Ser10), rabbit polyclonal (Millipore) at 4° C. overnight. After washing with PBST, primary antibodies were detected by Alexa Fluor-555 goat anti-rabbit immunoglobulin (IgG). Alexa Fluor 488 phalloidin (Invitrogen) and/or 4′,6-Diamidino-2-phenylindole (Sigma) were added as needed.

Immunostaining for Xenopus was performed as previously described (Lee et al., 2008). Embryos at stage 35-36 were fixed in 1×MEMFA. 12/101 (1:500; DSHB), and primary antibodies were detected with Alexa Fluor-488 or 555 goat anti-mouse Immunoglobulin (IgG).

### Mass Spectrometry

HUVECs were treated with 1% DMSO or 1% DMSO, 250 μM TBZ for 24 hours and lysed by Dounce homogenization in low salt buffer (10 mM Tris-HCl, pH 8.8, 10 mM KCl, 1.5 mM MgCl₂) with 0.5 mM DTT and protease inhibitor mixture (Calbiochem). 2,2,2-trifluoroethanol was added to 50% (v/v) for each sample, and samples were reduced with 15 mM DTT at 55° C. for 45 min and then alkylated with 55 mM iodoacetamide at room temperature for 30 min. Following alkylation, samples were diluted in digestion buffer (50 mM Tris-HCl, pH 8.0, 2 mM CaCl₂) to a final 2,2,2-trifluoroethanol concentration of 5% (v/v) and digested using proteomics grade trypsin (Sigma) at 1:50 (enzyme/protein) concentration and incubated at 37° C. for 4-5 h. Digestion was halted with the addition of 1% formic acid (v/v), and sample volume was reduced to 200 μl by SpeedVac centrifugation prior to loading on HyperSep C-18 SpinTips (Thermo). Samples were eluted (60% acetonitrile, 0.1% formic acid), reduced to 10 μl by SpeedVac centrifugation, and resuspended in sample buffer (5% acetonitrile, 0.1% formic acid). Tryptic peptides were then filtered through Microcon 10-kDa centrifugal filters (Millipore), and collected as flow-through. Peptides were chromatographically separated on a Zorbax reverse-phase C-18 column (Agilent) via a 230 min 5-38% acetonitrile gradient, then analyzed by on-line nanoelectrospray-ionization tandem mass spectrometry on an LTQ-Orbitrap (Thermo Scientific). Data-dependent ion selection was performed, collecting parent ion (MS1) scans at high resolution (60,000) and selecting ions with charge >+1 for collision-induced dissociation fragmentation spectrum acquisition (MS2) in the LTQ, with a maximum of 12 MS2 scans per MS1. Ions selected more than twice in a 30 sec window were dynamically excluded for 45 sec. MS2 spectra were interpreted using SEQUEST (Proteome Discoverer 1.3, Thermo Scientific), searching against human protein-coding sequences from Ensembl release 64⁴². Search results were then processed by Percolator³¹ at a 1% false discovery rate. Protein groups were generated comprising proteins with identical peptide evidence, omitting those proteins whose observed peptides could be entirely accounted for by other proteins with additional unique observations. Differential expression of proteins across TBZ-treated and control samples was quantified from the MS2 spectral count data using the APEX method of relative quantification³².

### Western Blotting and ELISA

HT1080 (2×10⁵ or 4×10⁴cells) were cultured in 6-well plates and treated with 1% DMSO or 1% DMSO, 250 μM TBZ for 24 hours. Cells were lysed in cell lysis buffer (Cell Signaling Technology) containing 1 mM PMSF, and analyzed by SDS-PAGE and western blotting using anti-VEGF (Santa Cruz, A-20) or anti-GAPDH (Cell Signaling Technology) antibodies. The secreted VEGF level in culture media was determined by enzyme-linked immunosorbent assay (ELISA; R&D) according to the manufacturer's instructions.

### Imaging and Image Analysis

Immunohistochemistry and kdr:GFP transgenic Xenopus laevis were imaged on an inverted Zeiss LSM5 Pascal confocal microscope and Zeiss 5-LIVE Fast Scanning confocal microscope. Confocal images were processed and cropped in Imaris software (BITPLANE) and Adobe Illustrator and Adobe Photoshop for compilation of Figures.

### EXAMPLE 2

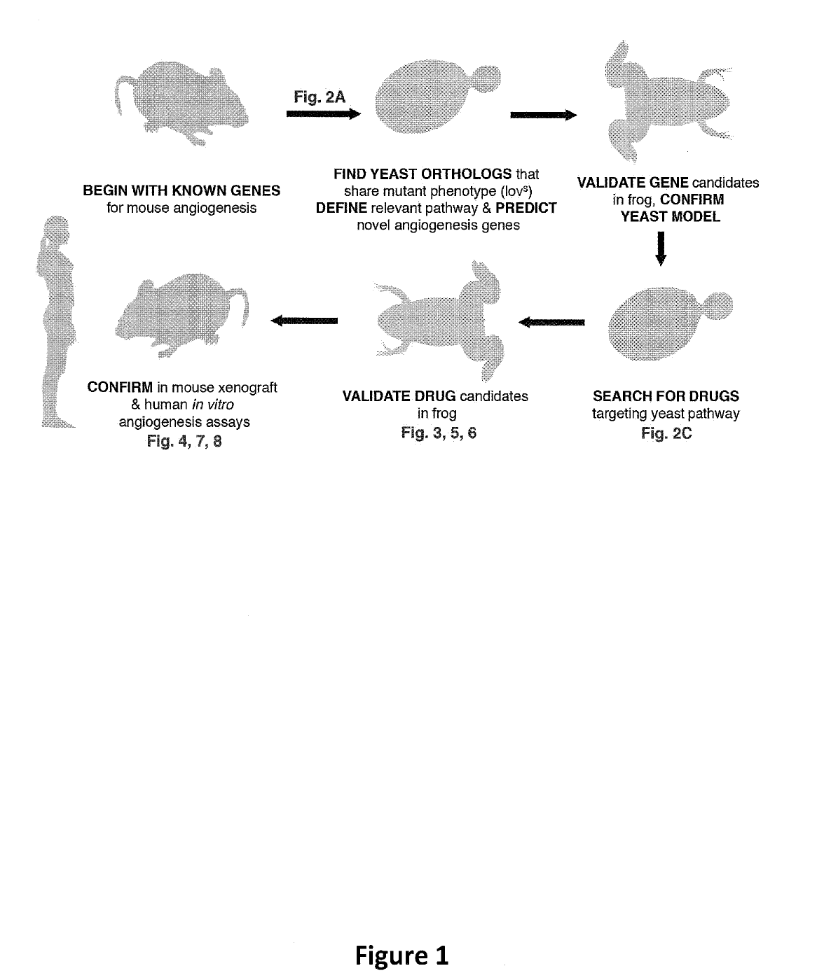

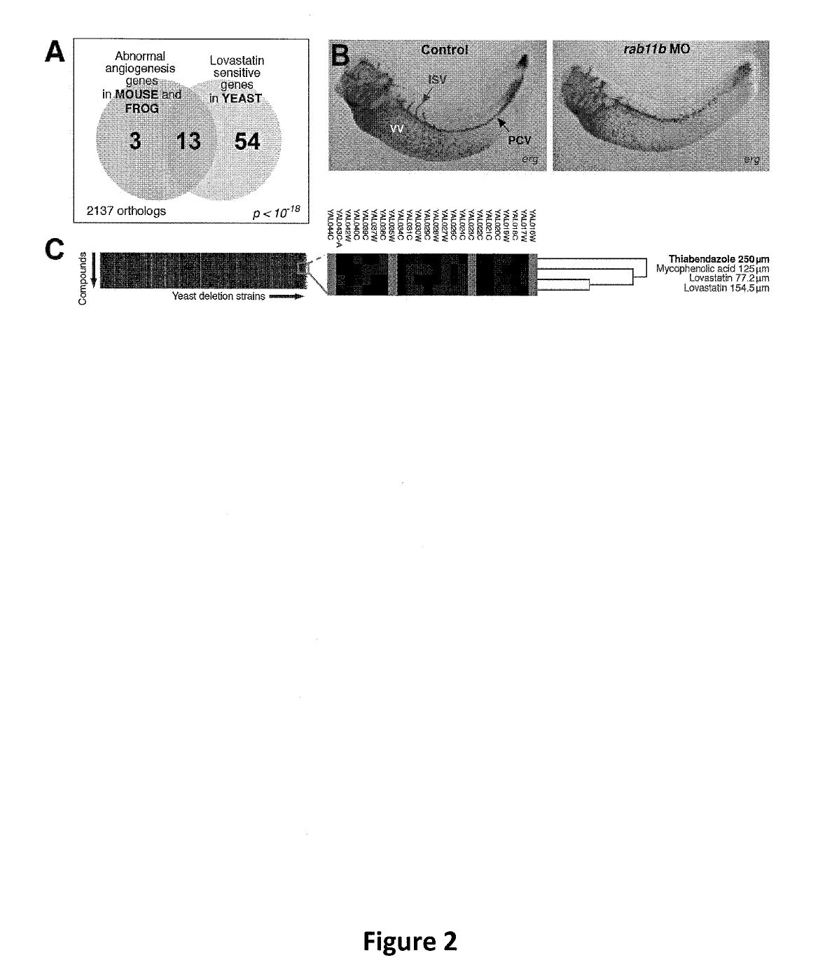

In the course of systematically identifying genetic modules that have been repurposed during the course of evolution (referred to as “phenologs”), we identified a conserved module that is relevant to lovastatin sensitivity in yeast and is also responsible for regulating angiogenesis in vertebrates (Ref. 8 and FIGS. 1, 2A). This pathway in yeast cells, which have neither blood nor blood vessels, suggested several candidate angiogenesis genes (Table 1).

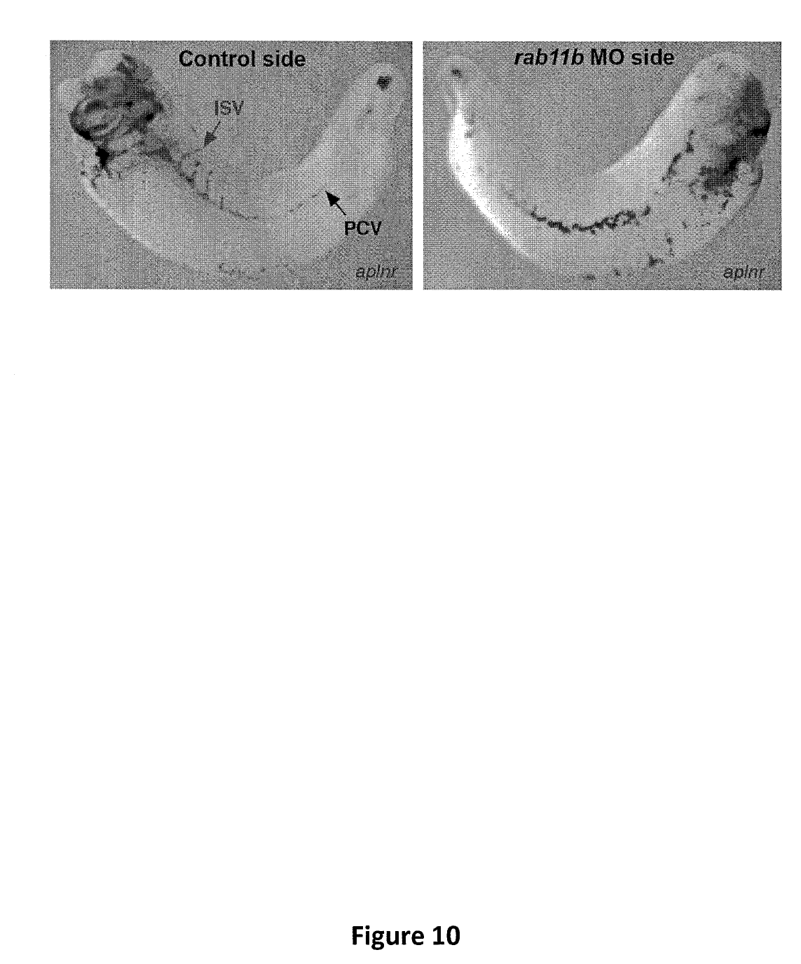

TABLE 1
Conserved genes in the vertebrate angiogenesis defect/yeast
lovastatin sensitivity gene module. Bold text indicates vertebrate
genes whose angiogenesis roles were known or confirmed by
the literature; italic text indicates genes whose roles were predicted
in ref.⁸ and confirmed in frogs and HUVEC cells in ref.⁸
and FIGS. 2B, 10.

| Human gene(s) | Yeast gene(s) |
|---|---|
| MAPK7 | SLT2 |
| MAP2K1 | PBS2 |
| MAPK14 | HOG1 |
| PPP3R1 | CNB1 |
| PSMA (FOLH1) | VPS70 |
| HMGCR | HMG1,2 |
| SIRT1 | HST1 |
| CSNK2A1 | CKA1 |
| SOX13 | IXR1 |
| RAB11B | YPT31 |
| HMHA1 | SAC7/BAG7 |
| TCEA1/3 | DST1 |
| TBL1XR1 | SIF2 |

We confirmed vascular expression for many of these genes in the frog, Xenopus, which provides an accurate, rapid, and tractable model vertebrate for in vivo studies of angiogenesis⁹⁻¹². In addition, we confirmed the function of several of the genes using knockdowns in frogs in vivo and in human umbilical vein endothelial cells (HUVECs) in vitro (Ref. 8 and FIGS. 2B, 10). In all, 13 genes were linked both to the growth phenotypes in yeast and to angiogenesis phenotypes in vertebrates (FIG. 2A; Table 1).

### EXAMPLE 3

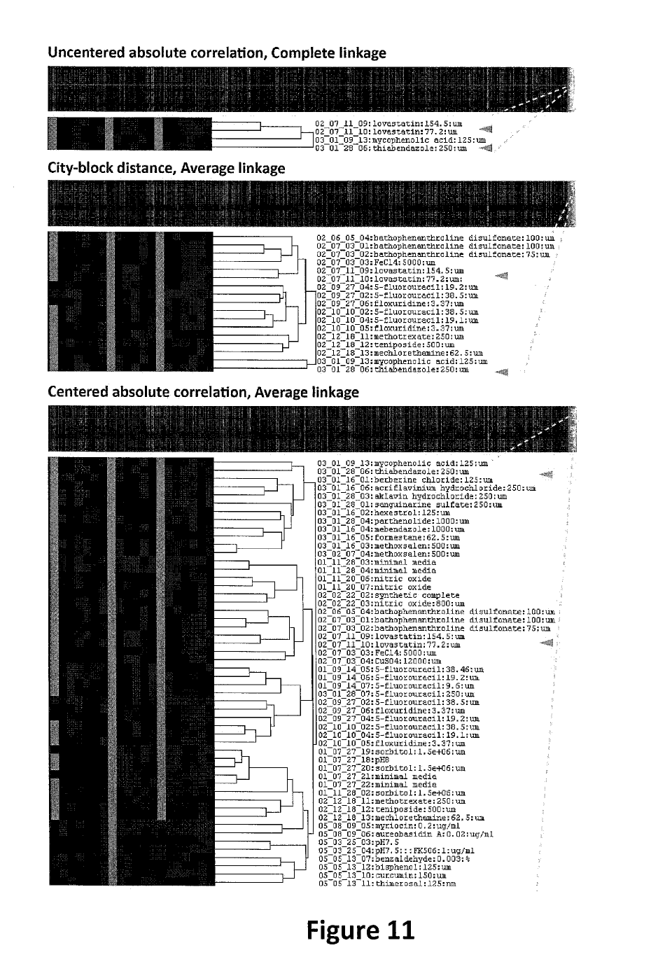

The remarkable conservation of this genetic module during evolution led us to test the possibility that small molecule inhibitors targeting the yeast pathway might also act as angiogenesis inhibitors. Indeed, lovastatin itself is known to inhibit angiogenesis^{13,14} and may reduce the incidence of melanoma^{15,16}. We devised a strategy to exploit the evolutionary repurposing of the module in order to direct our search (FIG. 1). Additional compounds were computationally prioritized based upon their measured synthetic genetic interactions with the 13 yeast genes¹⁷ using clustering algorithms to identify compounds with genetic interaction profiles similar to that of lovastatin (FIGS. 2C, 11; Table 2).

TABLE 2
Compounds computationally prioritized as candidate angiogenesis
effectors. 19 alternate hierarchical clustering trials were performed varying
the choice of clustering algorithm and the measure of similarity between
drug-gene interaction profiles (from ref. 17), as described in the Materials
and Methods, and compounds were selected by the frequency with which
they occured in the same subcluster as lovastatin.

| | | Number of |
|---|---|---|
| | Known | hierarchical clustering |
| | effects | trials within |
| | on | two branches of |
| Compound | angiogenesis | lovastatin |
| FeCl₄ | | 16 |
| Bathophenathroline | | 15 |
| disulfonate | | |
| CuSO₄ | Activators^{‡} | 5 |
| Nitric oxide | Activator^{#} | 3 |
| Mycophenolic acid | Inhibitor* | 3 |
| Thiabendazole | | 2 |
| 5-fluorouracil | Inhibitor^{†} | 1 |
| Floxuridine | | 1 |

^{‡}A. Parke, P. Bhattacherjee, R. M. Palmer, N. R. Lazarus, Am J Pathol 130, 173 (1988).
^{#}D. G. Duda, D. Fukumura, R. K. Jain, Trends Mol Med 10, 143 (2004).
*S. Domhan et al., Mol Cancer Ther 7, 1656 (2008).
^{†}T. Browder et al., Cancer Res 60, 1878 (2000).

One compound—thiabendazole (TBZ; 4-(1H-1,3-benzodiazol-2-yl)-1,3-thiazole) was further investigated as described in the below experiments.

### EXAMPLE 4

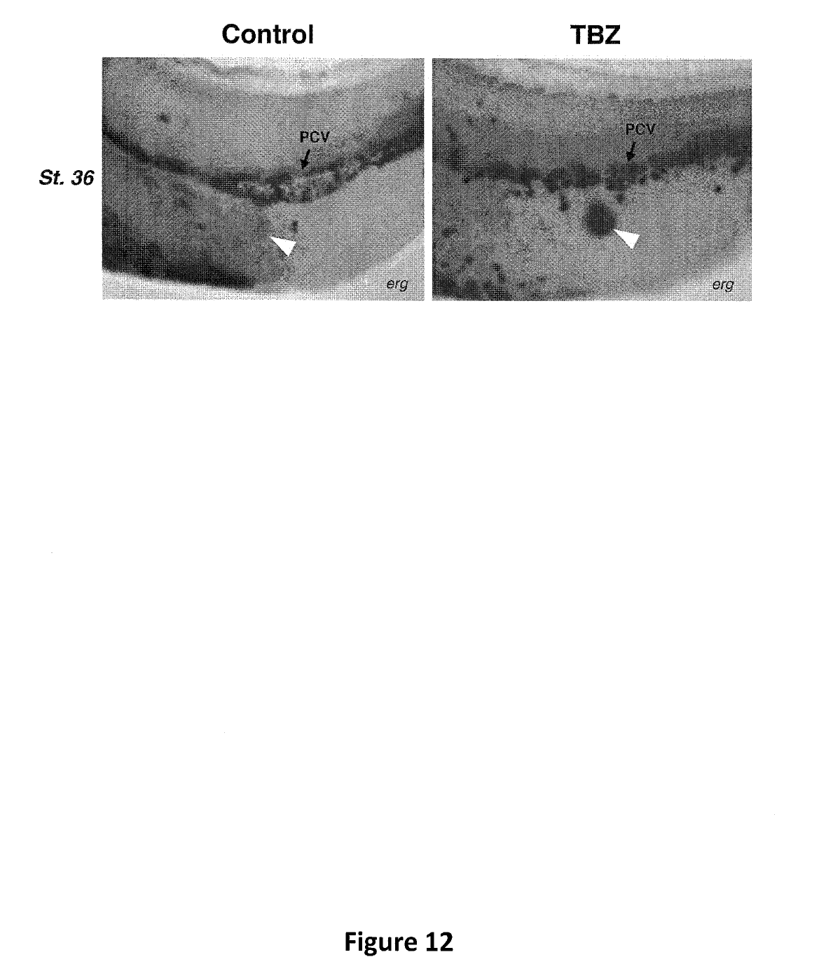

We tested the effect of TBZ on the expression of vascular-specific genes in developing Xenopus embryos in vivo. Using both the apelin-receptor (apinr) and the vascular ETS factor (erg), we found that TBZ treatment severely impaired angiogenesis (FIG. 3A-D). This result was confirmed in living embryos in which vasculature can be visualized by expression of GFP under control of a kdr enhancer/promoter fragment¹² (FIG. 3E-F). The receptor tyrosine kinase kdr is expressed in vascular endothelial cells, including both arteries and veins. In these assays, TBZ disrupts both types of vasculature. This activity was conserved in humans, as TBZ also inhibited angiogenesis in a dose-dependent manner in HUVECs in vitro (FIG. 4). We then sought to position the site of TBZ action relative to that of VEGF, as this growth factor is central to both normal and pathogenic angiogenesis^{2,3}. In frog embryos, ectopic VEGF potently induces ectopic angiogenesis⁹, and this effect was blocked by TBZ, suggesting that the drug acts downstream of this key regulatory node (FIG. 12).

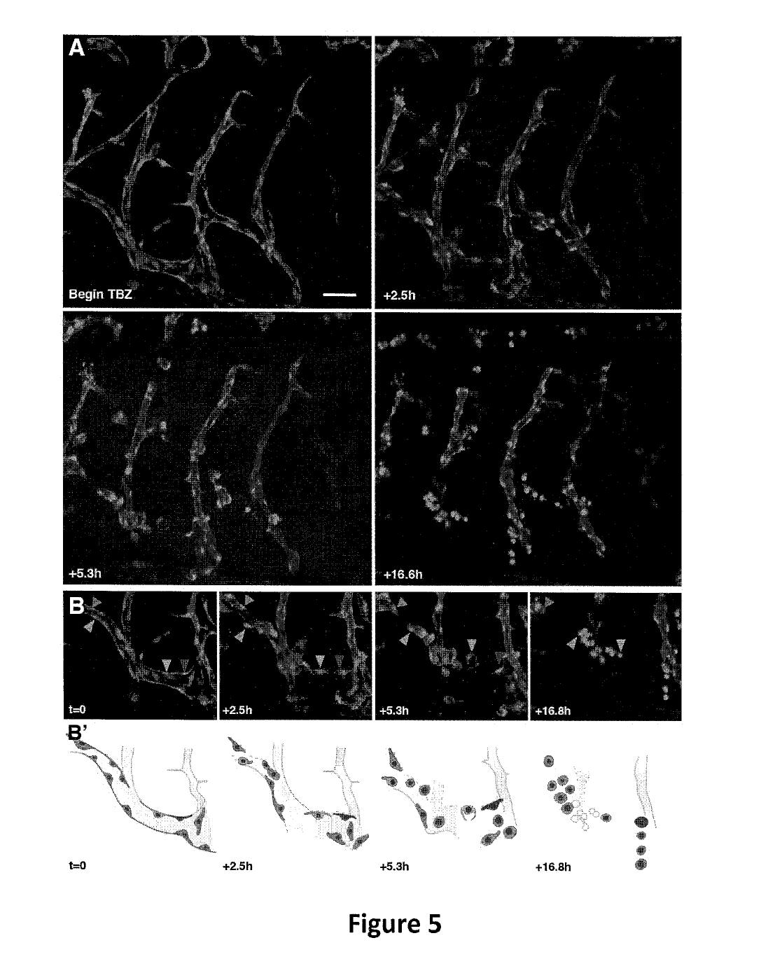

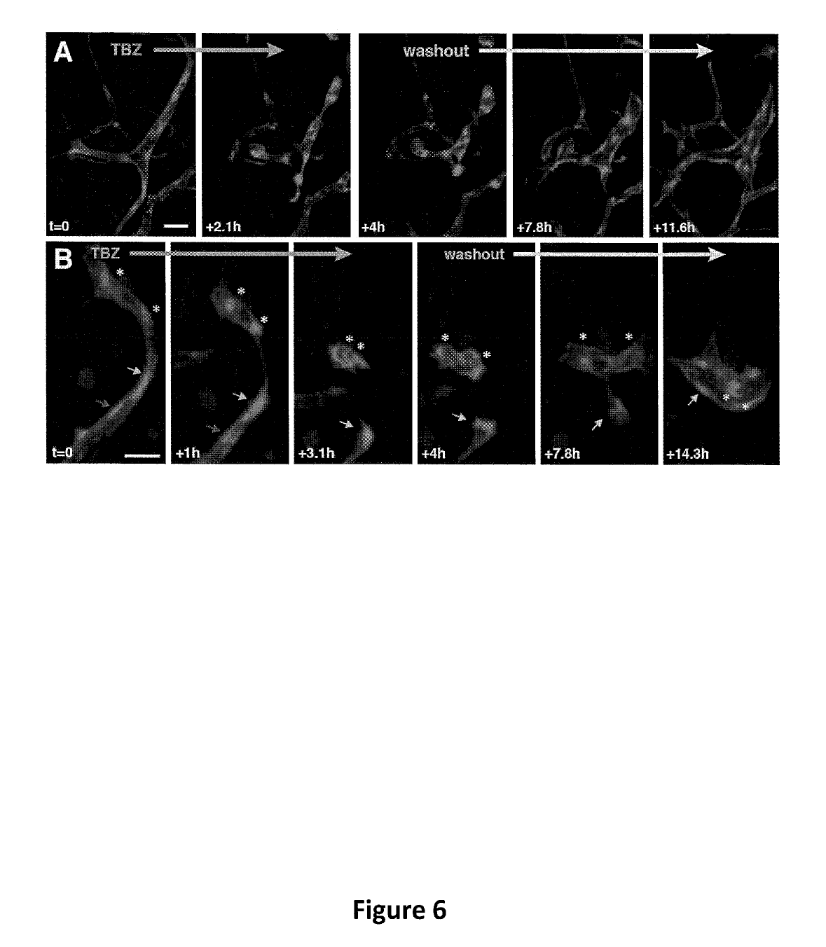

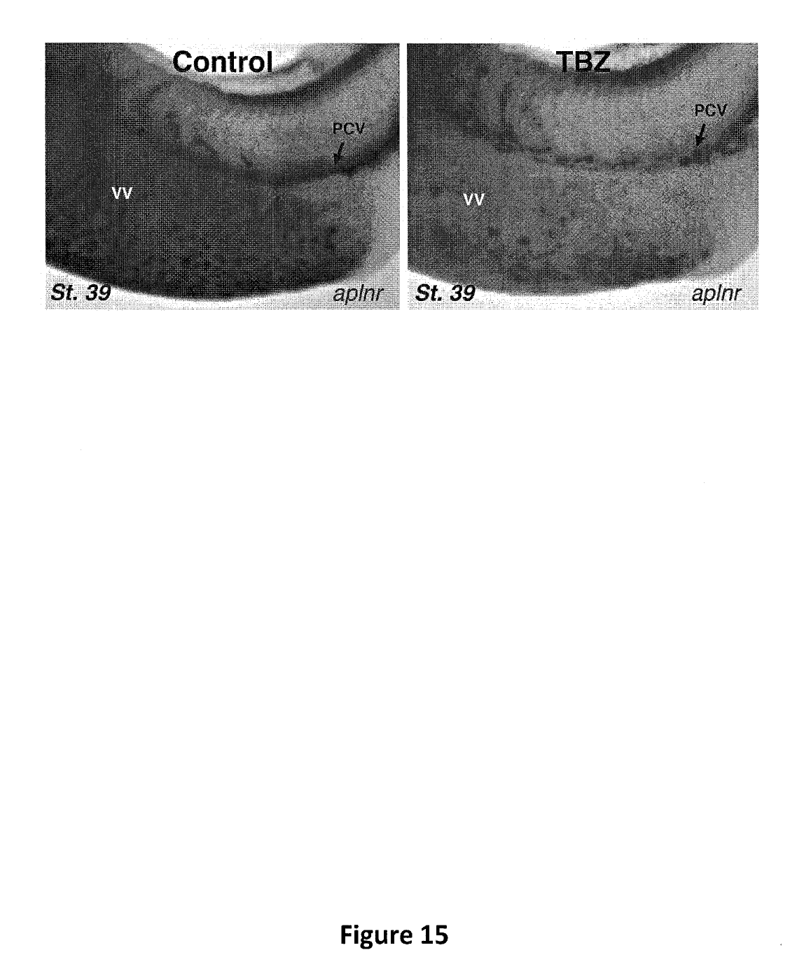

To analyze action of TBZ precisely on inhibition of angiogenesis and disruption of vasculature, we tested TBZ on two different developmental stages. Xenopus late tailbud embryos at stage 31 were selected for testing the inhibitory effect of TBZ on angiogenesis (FIG. 3A-D). This embryonic stage coincides with the onset of intersomitic vein (ISV) angiogenesis, the process of forming smaller blood vessels by sprouting from pre-existing blood vessels. To observe disruption of existing vasculature, we selected stage 35/36 embryos in which the posterior cardinal vein (PCV) and ISV are already developed (FIG. 15). In addition, in vivo disruption of newly established vasculature was visualized using time-lapse fluorescence microscopy of the vasculature, where an increased retraction and/or increased rounding of vascular endothelial cells (compared to a control) indicates disruption of newly established vasculature, as shown in FIGS. 5, 6.

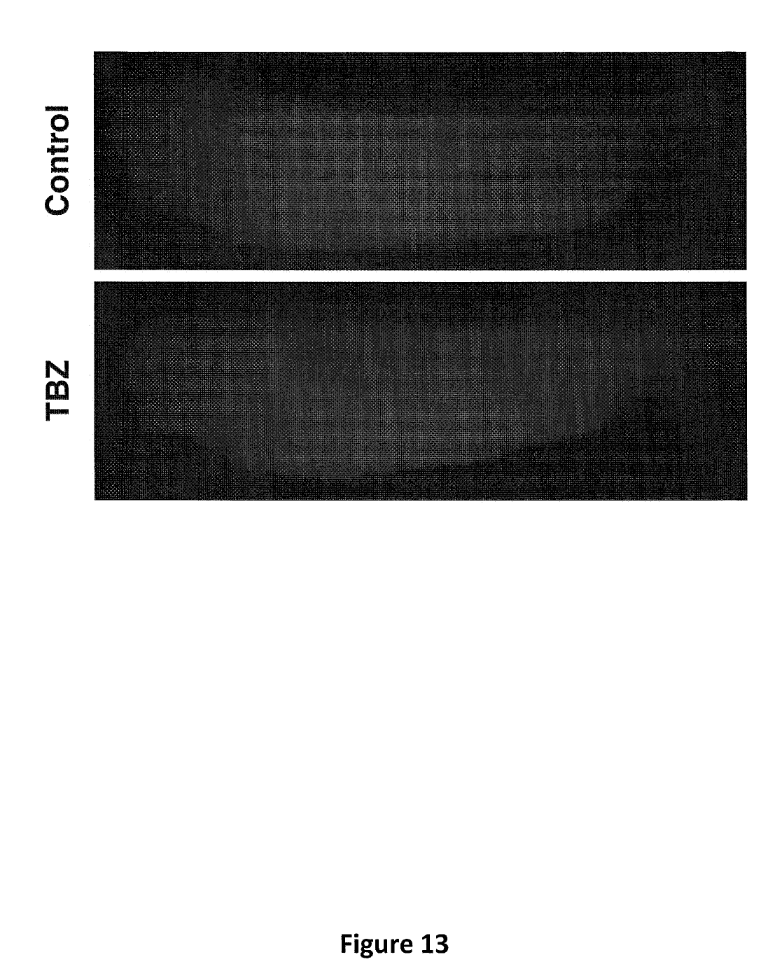

These data implicate TBZ as an effective inhibitor of angiogenesis. Importantly, we observed angiogenesis inhibition in both human cells in vitro and in Xenopus embryos in vivo at a concentration of 100-250 μM. This dose corresponds to 20-50 mg/kg (FIGS. 3, 4), which is notable because the oral LD50 of MINTEZOL is 1.3-3.6 g/kg, 3.1 g/kg and 3.8 g/kg in the mouse, rat, and rabbit, respectively, and the human approved recommended maximum daily dose is 3 grams, corresponding to 50 mg/kg for 60 kg patients. Finally, we note that the overall morphology and patterning of TBZ treated Xenopus embryos was grossly normal at the stages when the vasculature was severely disrupted (FIG. 13). Consistent with this, TBZ has good safety data in humans and model animals at the doses for which we observe a specific inhibition of angiogenesis^{18,19}.

### EXAMPLE 5

We next asked if angiogenesis inhibition may be a general property of benzimidazoles. Examination of commercially-available TBZ derivatives showed that this is not the case, with benzimidazole itself inactive in reducing angiogenesis at doses up to 1 mM and administration of other benzimidazoles causing diverse developmental defects but not angiogenesis inhibition (FIG. 14). These findings are thus significant for demonstrating a high level of precision for this evolutionary approach to drug discovery.

### EXAMPLE 6

We next sought to better understand the cellular basis for angiogenesis inhibition by TBZ. In the course of our experiments, we noted an interesting feature of the vasculature in TBZ treated embryos: disconnected and scattered arrays of cells in which vascular gene expression persisted (FIGS. 3B, D; 12). Hypothesizing that such morphological defects in the absence of changes to vascular cell fates may stem from impairment of junctional integrity in the vasculature, we tested the ability of TBZ to disrupt pre-existing vasculature by treatments at later stages. While not intending to limit the invention to any particular mechanism, TBZ treatment elicited overt breakdown of established vasculature at these stages (FIG. 15).

### EXAMPLE 7

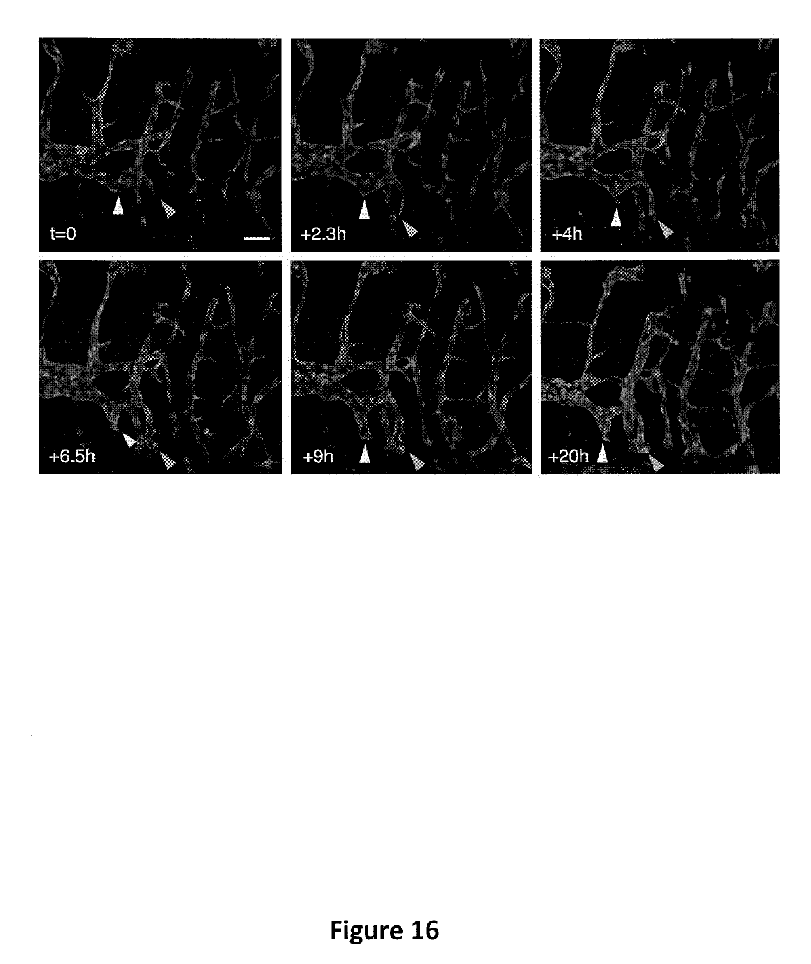

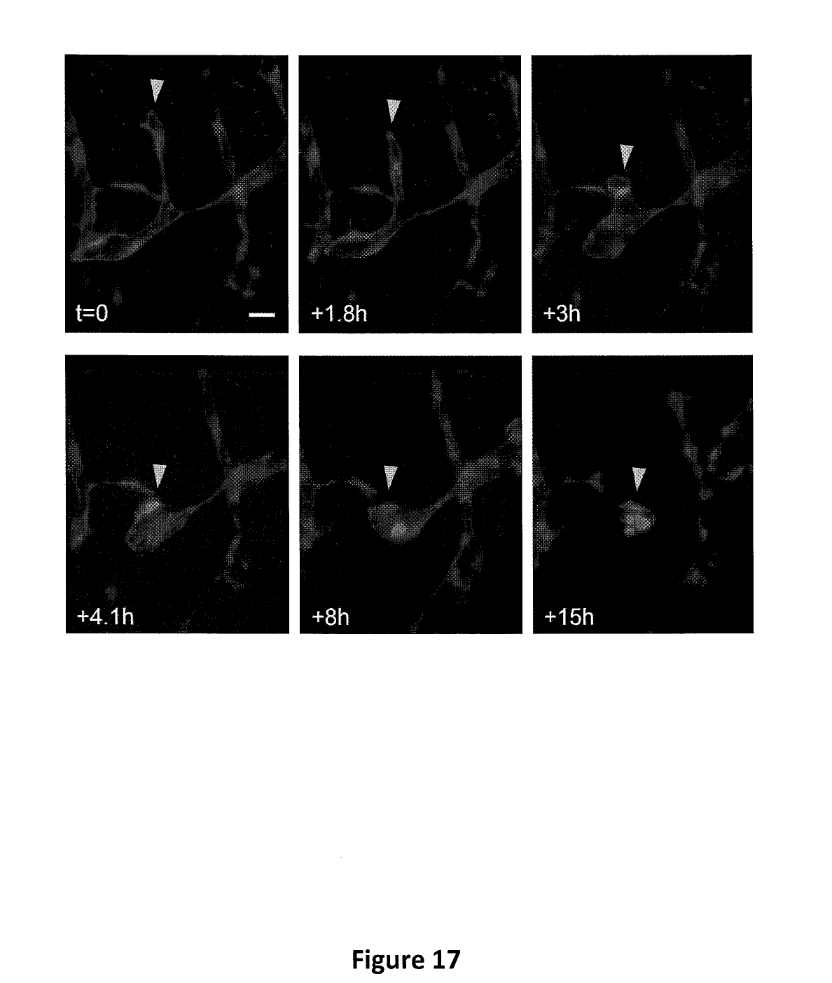

As a direct test of the vascular disrupting activity of TBZ, we performed time-lapse imaging of developing vasculature. Using Kdr-GFP transgenic embryos¹² and time-lapse confocal microscopy²², we could effectively image developing vasculature in vivo for periods of up 20 hours. During this time, the growth of existing blood vessels and the sprouting of new vasculature could be easily followed (FIG. 16). Treatment with TBZ completely prevented growth and sprouting of blood vessels, and moreover elicited a striking disintegration of established blood vessels after approximately 90 minutes of exposure (FIGS. 5A; 17). Upon longer exposures, endothelial cells scattered and many underwent dramatic rounding (FIGS. 5A, 17). These data demonstrate the efficacy of TBZ as a vascular disrupting agent.

### EXAMPLE 8

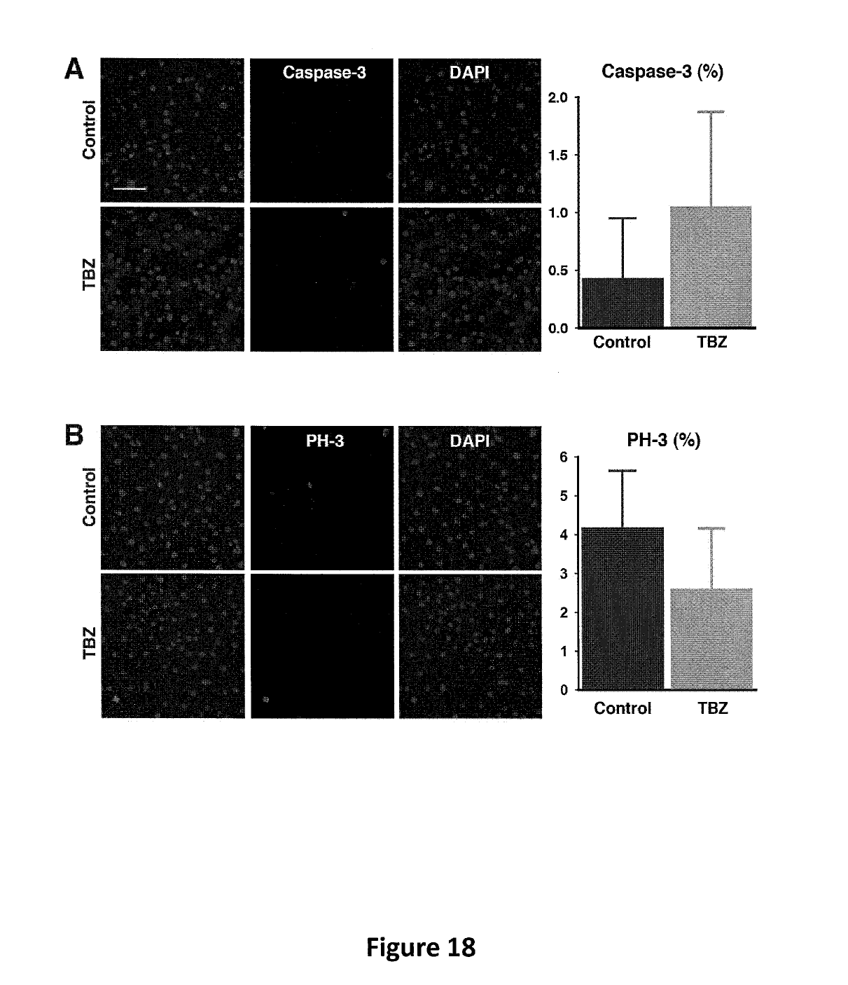

Previously-defined VDAs can act either by targeting endothelial cells for selective cell death (e.g. ASA404, Ref. 23) or by disrupting endothelial cell-cell junctions (e.g. combrestatin A4, Ref. 24), and so we sought to distinguish between these two possible mechanisms for TBZ action. We noted that treatment with TBZ doses sufficient to block angiogenesis elicited only modest increases in apoptosis in cultured HUVECs (FIG. 18). Moreover, vascular gene expression in dispersed, rounded kdr-GFP+ endothelial cells in vivo reliably persisted for up to 17 hours after TBZ treatment, a result that is consistent with the persistence of apinr and erg expression (FIGS. 3B, D; 12). These data argue against a role for apoptosis in vascular disruption by TBZ.

### EXAMPLE 9

To test more directly the above findings against a role for apoptosis in vascular disruption by TBZ, we performed washout experiments. Compellingly, washout of the drug after overt TBZ-induced cell dispersal and rounding resulted in significant re-spreading of endothelial cells and re-formation of blood vessels in vivo (FIGS. 5B; 6). In several cases, widely-separated kdr-GFP-positive endothelial cells reconnected into small, nascent blood vessels after washout of TBZ (FIG. 6). While not intending to limit the invention to any particular mechanism, together, these data demonstrate that the ability of TBZ to disrupt established vasculature is independent of cell death and suggest that instead the drug acts at the level of endothelial cell-cell adhesion.

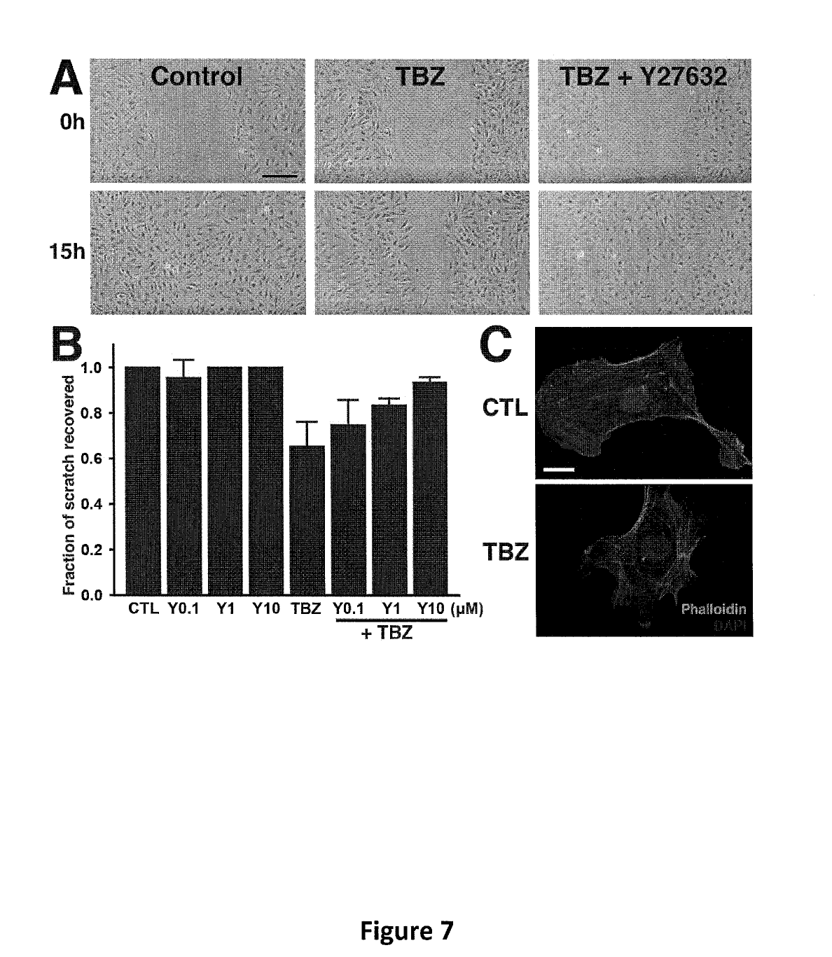

The data above suggest that the mechanism of TBZ action distinguishes it from VDAs such as ASA404, which act by inducing endothelial cell apoptosis²³, but which failed to show efficacy in a recent Phase III clinical trial for treatment of lung cancer²⁵. Finally, we found that treatment with TBZ significantly slowed endothelial cell migration in a scratch wound assay using cultured HUVECs (FIGS. 7AB). This quantitative in vitro assay with mammalian cells, combined with our in vivo data from Xenopus, demonstrate that TBZ disrupts established vasculature not by eliciting cell death but rather by perturbing endothelial cell behavior.

### EXAMPLE 10

The effect of TBZ on endothelial cells is striking and rapid. Our in vivo imaging of the vasculature revealed that endothelial cells retract from one another and round up within 2 hours of TBZ treatment (FIGS. 5, 6). Moreover, we observed that this effect is reversible by washout within a similarly rapid time frame (FIG. 6). The rapid time-frames observed here argue that TBZ may act at the level of the cytoskeleton to influence endothelial cell behavior.

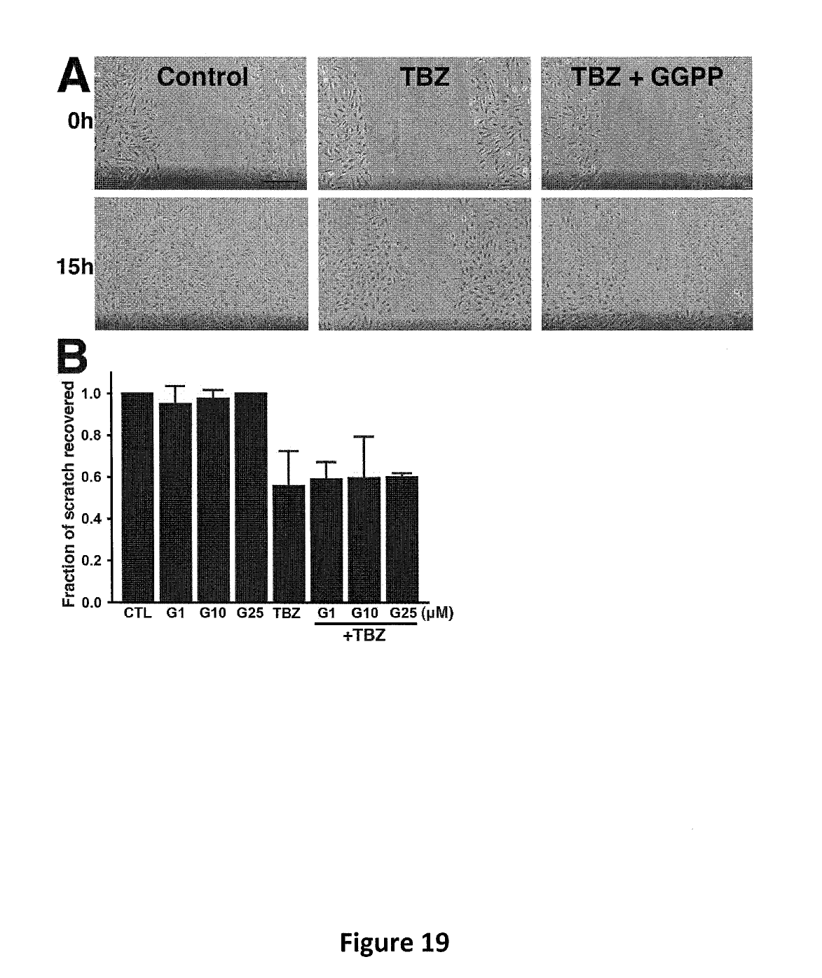

We first considered that, while not an assumption of the phenolog approach (see ref. 8), TBZ may nonetheless impact the vasculature by the same mechanism as lovastatin. Lovastatin disrupts angiogenesis at least in part by perturbing the geranyl-geranylation of the RhoA GTPase, thereby abrogating its activity¹⁴. RhoA is a critical regulator of actin-based behaviors in all animal cells³³, and the loss of RhoA signaling in endothelial cells treated with lovastatin is directly linked to cytoskeletal changes and inhibition of angiogenesis¹⁴. Indeed, inhibition of angiogenesis by lovastatin can be overcome by addition of geranyl-geranyl pyrophosphate (GGPP; ref. 13, 14). We therefore used the HUVEC scratch-wound closure model to quantitatively assess the effects of GGPP addition on TBZ action. However, we found that addition of GGPP did not reverse the action of TBZ on HUVEC cell motility in this assay (FIG. 19). Similarly, while TBZ has been observed to affect the activity of porcine heart mitochondria³⁴, we detected no differences in mitochondrial mass (measured by MitoTracker Green signal) or mitochondrial membrane potential (measured as the ratio of MitoTracker Red signal to Mitotracker Green signal), thus ruling out this potential activity as being relevant.

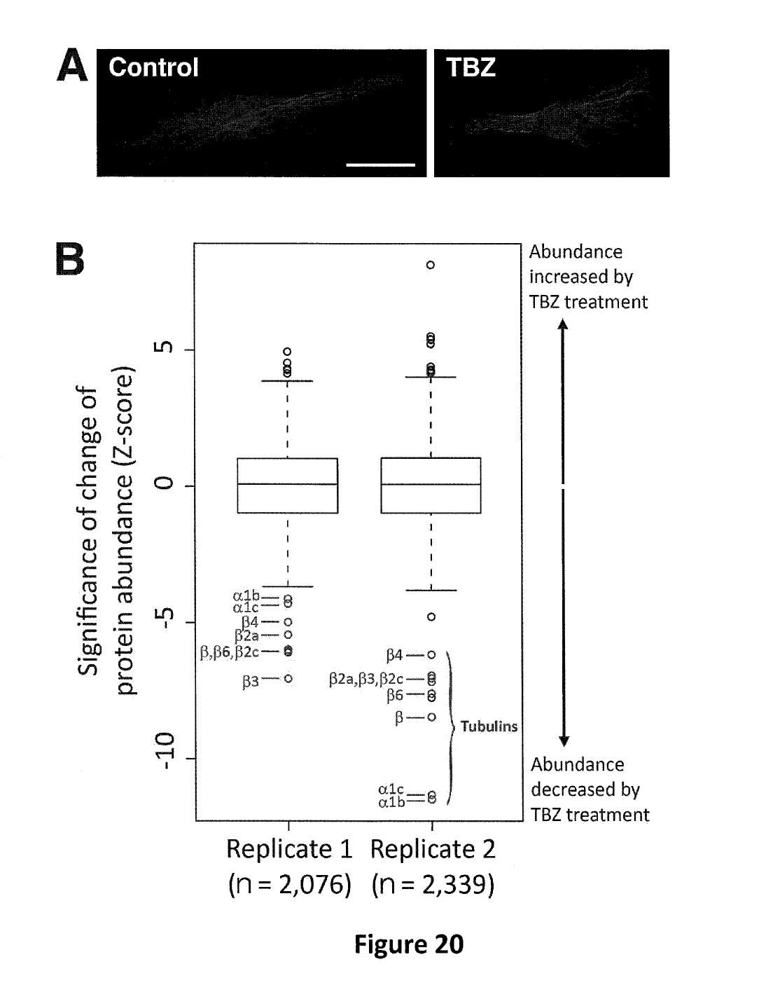

We next considered the possibility that TBZ acted on the vasculature at the level of the microtubule (MT) cytoskeleton, because TBZ has been found to disrupt microtubule assembly and dynamics in a number of cell types (e.g. ref. 35-37), and because several currently-studied VDAs act as MT-disrupting agents^{20,38}. Curiously, TBZ had only a very slight effect on the gross organization of the MT cytoskeleton in HUVEC cells in culture (FIG. 20A), but a quantitative analysis using mass-spectrometry revealed a significant reduction in the abundance of several tubulin proteins following treatment of HUVECs with TBZ (FIG. 20B).

Many MT-targeting VDAs act via hyper-activation of Rho signaling^{24,39,40}, likely reflecting the key role of MT-binding RhoGEFs⁴¹. We reasoned, therefore, that TBZ may also act via increased Rho signaling, as the drug elicited several phenotypes known to be associated with dysregulated Rho signaling (e.g. cell rounding, re-distribution of actin filaments, and defects in cell motility; FIG. 7). To test this model directly, we asked if disruption of Rho signaling might counteract the effects of TBZ. Indeed, pharmacological disruption of Rho kinase function using the small molecule Y27632 elicited a significant and dose-dependent rescue of the TBZ-induced HUVEC cell motility defect (FIGS. 7A, B). Together, these data suggest that vascular disruption by TBZ results from reduced tubulin levels and hyper-active Rho signaling.

### EXAMPLE 11

To determine if TBZ may be useful in the arena of cancer therapy, we tested the ability of TBZ to slow the growth of solid vascularized tumors in a mammal.

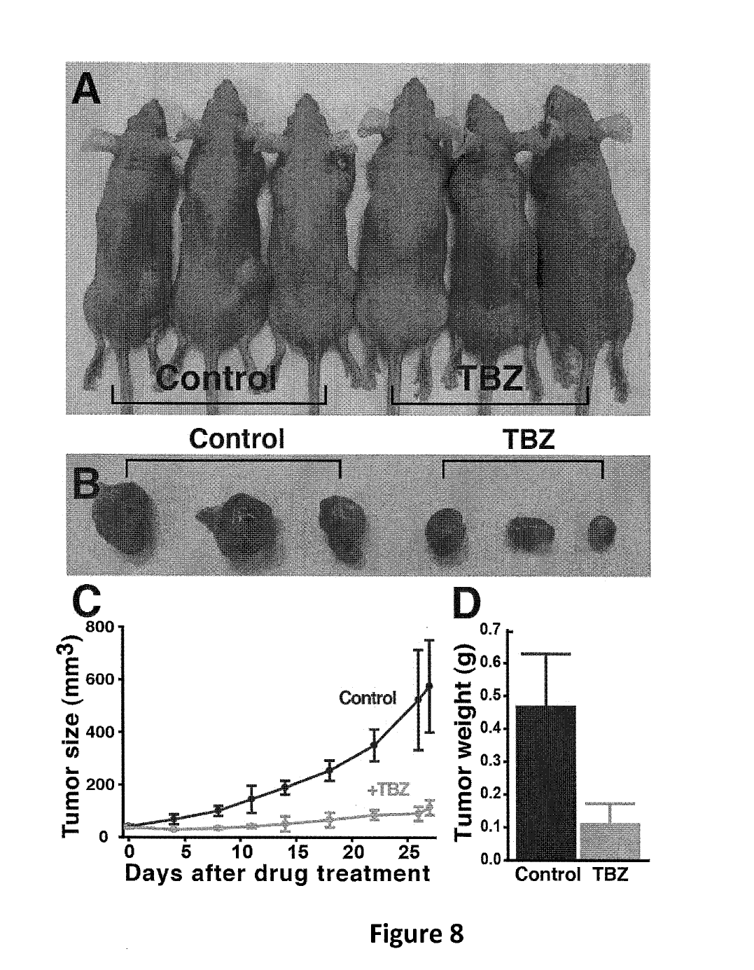

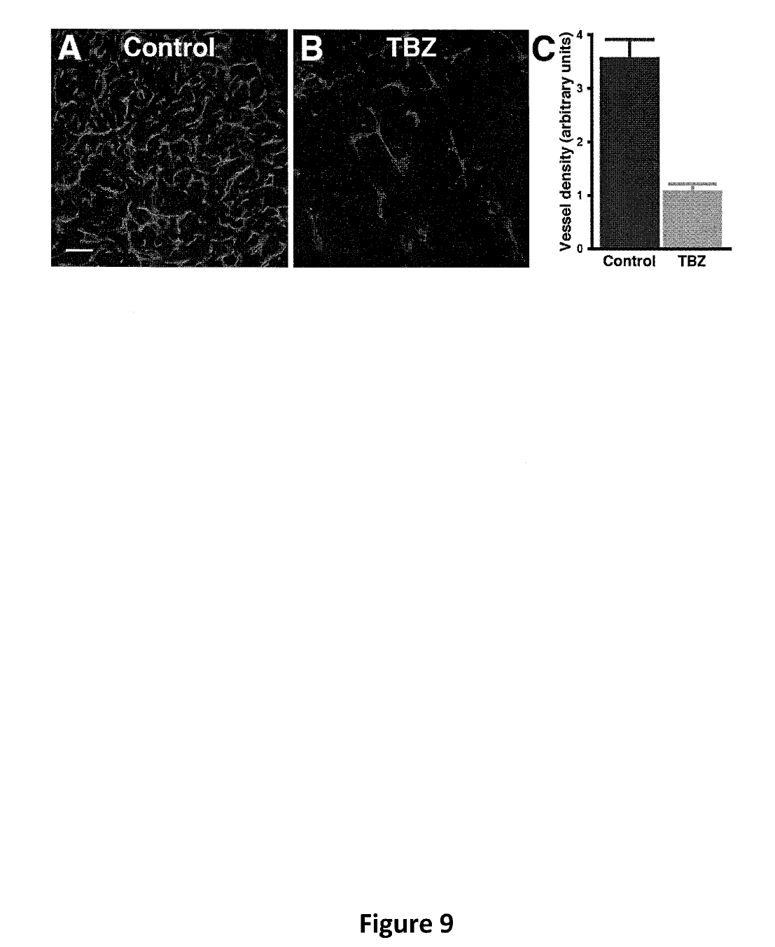

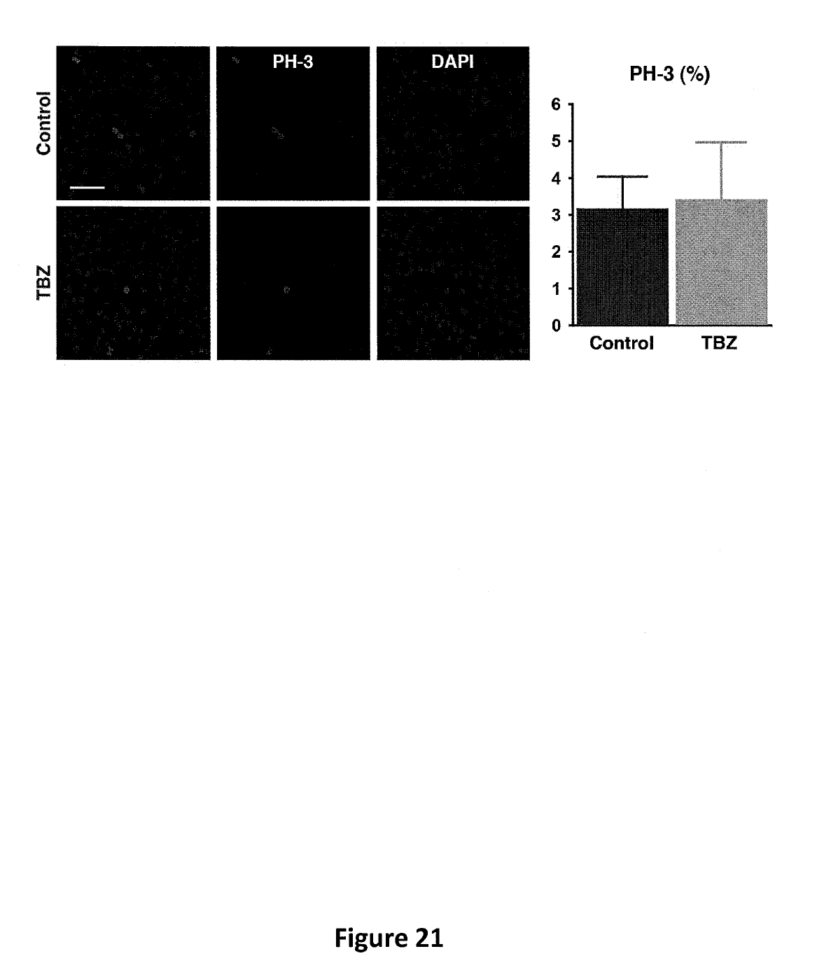

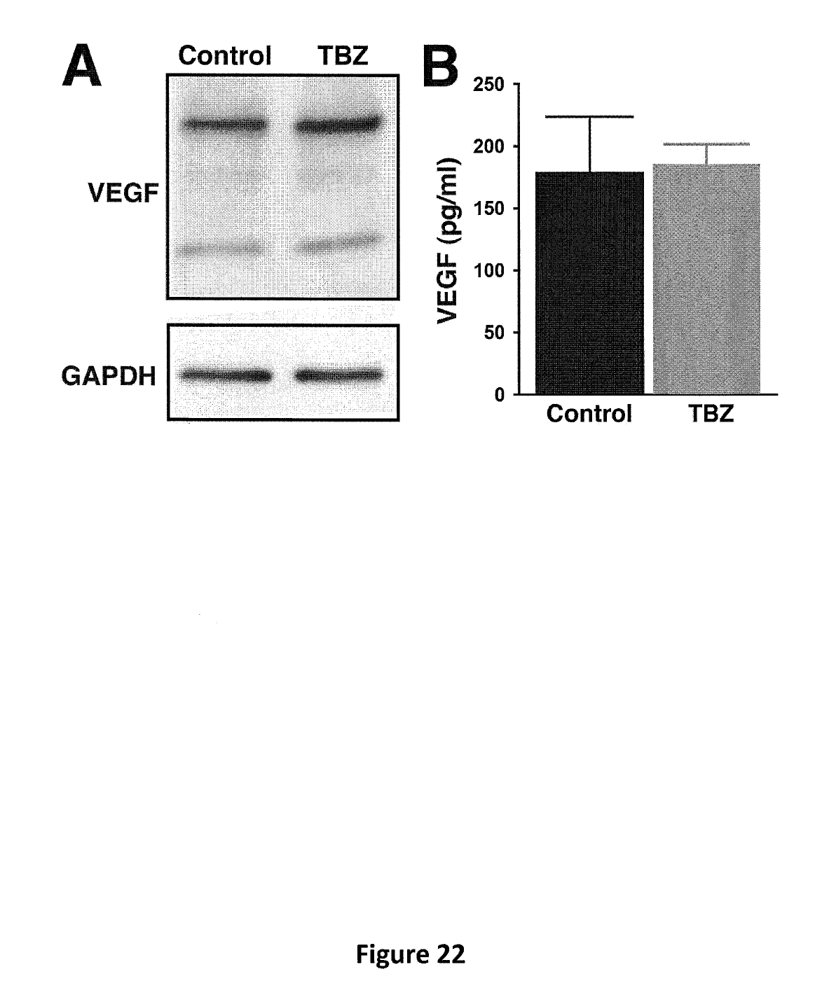

We employed a mouse xenograft model typical of those proven valuable in indicating the effectiveness of anti-angiogenesis therapy^{26,27}. We found that TBZ treatment significantly slowed HT1080 human fibrosarcoma xenograft growth in athymic Cre nu/nu mice²⁸, as assessed by a time course of tumor size and also by final tumor mass (FIG. 8). Our in vivo data from Xenopus, as well as our human in vitro data, suggest that TBZ likely slows tumor growth by acting at the level of the vasculature (FIGS. 3, 4). Consistent with this model, TBZ treatment did not alter the rate of proliferation in HT1080 cells when cultured in vitro but did significantly impair tumor microvessel density in xenografts (FIGS. 9, 21). In addition, we noted that treatment with TBZ did not alter the levels of VEGF expressed or secreted by HT1080 cells, consistent with it acting downstream of VEGF in tumor xenografts (FIG. 22), as it does in developing Xenopus embryos in vivo (FIG. 3). Notably, we employed a TBZ dose of 50 mg/kg for these experiments, which is concordant with the FDA-approved maximum recommended daily dose of TBZ in humans.

### REFERENCES

1. Carmeliet, P. & Jain, R. K. Molecular mechanisms and clinical applications of angiogenesis. Nature 473, 298-307 (2011).

2. Kerbel, R. S. Tumor angiogenesis. N Engl J Med 358, 2039-49 (2008).

3. Heath, V. L. & Bicknell, R. Anticancer strategies involving the vasculature. Nat Rev Clin Oncol 6, 395-404 (2009).

4. Folkman, J. Angiogenesis: an organizing principle for drug discovery? Nat Rev Drug Discov 6, 273-86 (2007).

5. Bondy, M. L. et al. Brain tumor epidemiology: consensus from the Brain Tumor Epidemiology Consortium. Cancer 113, 1953-68 (2008).

6. Chi, A. S., Sorensen, A. G., Jain, R. K. & Batchelor, T. T. Angiogenesis as a therapeutic target in malignant gliomas. Oncologist 14, 621-36 (2009).

7. Jain, R. K. et al. Angiogenesis in brain tumours. Nat Rev Neurosci 8, 610-22 (2007).

8. McGary, K. L. et al. Systematic discovery of nonobvious human disease models through orthologous phenotypes. Proc Natl Acad Sci USA 107, 6544-9 (2010).

9. Cleaver, O., Tonissen, K. F., Saha, M. S. & Krieg, P. A. Neovascularization of the Xenopus embryo. Dev Dyn 210, 66-77 (1997).

10. Ny, A., Autiero, M. & Carmeliet, P. Zebrafish and Xenopus tadpoles: small animal models to study angiogenesis and lymphangiogenesis. Exp Cell Res 312, 684-93 (2006).

11. Kalin, R. E., Banziger-Tobler, N. E., Detmar, M. & Brandli, A. W. An in vivo chemical library screen in Xenopus tadpoles reveals novel pathways involved in angiogenesis and lymphangiogenesis. Blood 114, 1110-22 (2009).

12. Doherty, J. R., Johnson Hamlet, M. R., Kuliyev, E. & Mead, P. E. A flk-1 promoter/enhancer reporter transgenic Xenopus laevis generated using the Sleeping Beauty transposon system: an in vivo model for vascular studies. Dev Dyn 236, 2808-17 (2007).

13. Depasquale, I. & Wheatley, D. N. Action of Lovastatin (Mevinolin) on an in vitro model of angiogenesis and its co-culture with malignant melanoma cell lines. Cancer Cell Int 6, 9 (2006).

14. Park, H. J. et al. 3-hydroxy-3-methylglutaryl coenzyme A reductase inhibitors interfere with angiogenesis by inhibiting the geranylgeranylation of RhoA. Circ Res 91, 143-50 (2002).

15. Shellman, Y. G. et al. Lovastatin-induced apoptosis in human melanoma cell lines. Melanoma Res 15, 83-9 (2005).

16. Splichal, J. E., Ornstein, D. L., Gia Hong-Dice, Y., Downs, J. R. & Fischer, J. R. Lovastatin for the prevention of melanoma: Analysis of AFCAPS/TexCAPS. Am Soc Clin Oncol Abstract, 1397 (2001).

17. Hillenmeyer, M. E. et al. The chemical genomic portrait of yeast: uncovering a phenotype for all genes. Science 320, 362-5 (2008).

18. Edwards, I. R., Ferry, D. G. & Temple, W. A. Fungicides and Related Compounds. in Handbook of Pesticide Toxicology, Vol. 3 Classes of Pesticides (eds. Hayes, W. J. & Laws, E. R.) (Academic Press, Inc., New York, 1991).

19. Gosselin, R. E., Smith, R. P. & Hodge, H. C. Clinical Toxicology of Commercial Products, (Williams and Wilkins, Baltimore, Md., 1984).

20. Tozer, G. M., Kanthou, C. & Baguley, B. C. Disrupting tumour blood vessels. Nat Rev Cancer 5, 423-35 (2005).

21. Hinnen, P. & Eskens, F. A. Vascular disrupting agents in clinical development. Br J Cancer 96, 1159-65 (2007).

22. Kieserman, E. K., Lee, C., Gray, R. S., Park, T. J. & Wallingford, J. B. High-magnification in vivo imaging of Xenopus embryos for cell and developmental biology. Cold Spring Harb Protoc 2010, pdb prot5427 (2010).

23. Ching, L. M. et al. Induction of endothelial cell apoptosis by the antivascular agent 5,6-Dimethylxanthenone-4-acetic acid. Br J Cancer 86, 1937-42 (2002).

24. Kanthou, C. & Tozer, G. M. The tumor vascular targeting agent combretastatin A-4-phosphate induces reorganization of the actin cytoskeleton and early membrane blebbing in human endothelial cells. Blood 99, 2060-9 (2002).

25. Lara, P. N., Jr. et al. Randomized phase III placebo-controlled trial of carboplatin and paclitaxel with or without the vascular disrupting agent vadimezan (ASA404) in advanced non-small-cell lung cancer. J Clin Oncol 29, 2965-71 (2011).

26. Crawford, Y. & Ferrara, N. VEGF inhibition: insights from preclinical and clinical studies. Cell Tissue Res 335, 261-9 (2009).

27. Couffinhal, T., Dufourcq, P., Barandon, L., Leroux, L. & Duplaa, C. Mouse models to study angiogenesis in the context of cardiovascular diseases. Front Biosci 14, 3310-25 (2009).

28. Wall, A. et al. Differentiation of angiogenic burden in human cancer xenografts using a perfusion-type optical contrast agent (SIDAG). Breast Cancer Res 10, R23 (2008).

29. Sive, H. L., Grainger, R. M. & Harland, R. M. Early Development of Xenopus laevis: A laboratory manual, (Cold Spring Harbor Press, Cold Spring Harbor, N. Y., 2000).

30. Bruhl, T. et al. Homeobox A9 transcriptionally regulates the EphB4 receptor to modulate endothelial cell migration and tube formation. Circ Res 94, 743-51 (2004).

31. Kall L, Canterbury J D, Weston J, Noble W S, MacCoss M J. Semi-supervised learning for peptide identification from shotgun proteomics datasets. Nat Methods 4, 923-925 (2007).

32. Lu P, Vogel C, Wang R, Yao X, Marcotte E M. Absolute protein expression profiling estimates the relative contributions of transcriptional and translational regulation. Nat Biotechnol 25, 117-124 (2007).

33. Jaffe A B, Hall A. Rho GTPases: biochemistry and biology. Annu Rev Cell Dev Biol 21, 247-269 (2005).

34. Zhou Q, Zhai Y, Lou J, Liu M, Pang X, et al. Thiabendazole inhibits ubiquinone reduction activity of mitochondrial respiratory complex II via a water molecule mediated binding feature. Protein Cell 2, 531-542 (2011).

35. Davidse L C. Benzimidazole fungicides: Mechanis of action adn biological impact. Ann Rev Phytopathol 24, 43-65 (1986).

36. Davidse L C, Flach W. Interaction of thiabendazole with fungal tubulin. Biochim Biophys Acta 543, 82-90 (1978).

37. Pisano C, Battistoni A, Antoccia A, Degrassi F, Tanzarella C. Changes in microtubule organization after exposure to a benzimidazole derivative in Chinese hamster cells. Mutagenesis 15, 507-515 (2000).

38. Schwartz E L. Antivascular actions of microtubule-binding drugs. Clin Cancer Res 15, 2594-2601 (2009).

39. Ren X, Dai M, Lin L P, Li P K, Ding J. Anti-angiogenic and vascular disrupting effects of C9, a new microtubule-depolymerizing agent. Br J Pharmacol 156, 1228-1238 (2009).

40. Bayless K J, Davis G E. Sphingosine-1-phosphate markedly induces matrix metalloproteinase and integrin-dependent human endothelial cell invasion and lumen formation in three-dimensional collagen and fibrin matrices. Biochem Biophys Res Commun 312: 903-913 (2003).

41. Birukova A A, Adyshev D, Gorshkov B, Bokoch G M, Birukov K G, et al. GEF-H1 is involved in agonist-induced human pulmonary endothelial barrier dysfunction. Am J Physiol Lung Cell Mol Physiol 290, L540-548 (2006).

42. Flicek P, Aken B L, Ballester B, Beal K, Bragin E, et al. Ensembl's 10th year. Nucleic Acids Res 38, D557-562 (2010).

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for inducing disruption of vasculature in a tissue in a mammalian subject in need of inducing disruption of vasculature in said tissue, comprising administering to said subject a therapeutic amount of thiabendazole (TBZ) that is effective to induce disruption of vasculature in said tissue.

2. The method of claim 1, wherein said administration is oral, and said therapeutic amount of TBZ is 200 mg/kg body weight or less.

3. The method of claim 2, wherein said therapeutic amount of TBZ is 100 mg/kg body weight or less.

4. The method of claim 2, wherein said administration is oral, and said therapeutic amount of TBZ is from more than 1 mg/kg body weight to 200 mg/kg body weight.

5. The method of claim 2, wherein said therapeutic amount of TBZ does not cause mutations in said subject.

6. The method of claim 2, wherein said therapeutic amount of TBZ does not cause birth defects in the offspring of said subject.

7. The method of claim 1, wherein said subject has a disease associated with increased angiogenesis in said tissue compared to normal tissue lacking said disease.

8. The method of claim 7, wherein said disease comprises cancer.

9. The method of claim 8, wherein said cancer comprises cancer cells that do not express a tumor suppressor gene.

10. The method of claim 8, wherein said administering reduces one or more symptoms of said disease.

11. The method of claim 1, wherein said therapeutic amount is not fungicidal and is not parasiticidal and is not bactericidal in one or more tissue of said subject.

12. A method for reducing angiogenesis in a tissue in a mammalian subject in need of reducing angiogenesis in said tissue, comprising administering to said subject a therapeutic amount of thiabendazole (TBZ) that is effective to reduce angiogenesis in said tissue.

13. The method of claim 12, wherein said administration is oral, and said therapeutic amount of TBZ is 200 mg/kg body weight or less.

14. The method of claim 13, wherein said therapeutic amount of TBZ is 100 mg/kg body weight or less.

15. The method of claim 13, wherein said administration is oral, and said therapeutic amount of TBZ is from more than 1 mg/kg body weight to 200 mg/kg body weight.

16. The method of claim 13, wherein said therapeutic amount of TBZ does not cause mutations in said subject.

17. The method of claim 13, wherein said therapeutic amount of TBZ does not cause birth defects in the offspring of said subject.

18. The method of claim 12, wherein said subject has a disease associated with increased angiogenesis in said tissue compared to normal tissue lacking said disease.

19. The method of claim 18, wherein said disease comprises cancer.

20. The method of claim 19, wherein said cancer comprises cancer cells that do not express a tumor suppressor gene.

21. The method of claim 18, wherein said administering reduces one or more symptoms of said disease.

22. The method of claim 11, wherein said therapeutic amount is not fungicidal and is not parasiticidal and is not bactericidal in one or more tissue of said subject.

23. A method for reducing one or more symptoms of cancer in a tissue in a mammalian subject in need thereof, comprising administering to said subject a therapeutic amount of thiabendazole (TBZ) that is effective to reduce one or more symptoms of cancer in said tissue.
